(19) United States
(12) Patent Application Publication
(10) Pub. No.: US 2023/0419855 A1
(43) Pub. Date: Dec. 28, 2023

(54) SIMULATOR FOR SKILL-ORIENTED TRAINING OF A HEALTHCARE PRACTITIONER

(71) Applicant: VRSim, Inc., East Hartford, CT (US)

(72) Inventors: Matthew WALLACE, South Windsor, CT (US); Sara BLACKSTOCK, Vernon, CT (US); Alejandro FUDGE, Vernon, CT (US); Zachary LENKER, Bloomfield, CT (US); Katerine ANDERSON, Vernon, CT (US); David ZBORAY, Trumbull, CT (US); Maggie-Anne VOLZ, Perry, IA (US); Jay POULIN, Manchester, CT (US); Joshua ARMOUR, Hartford, CT (US); Paul ONG, Bolton, CT (US); Matthew BENNETT, Milford, CT (US); Sandra Kay LANDON, Pella, IA (US)

(21) Appl. No.: 18/140,743

(22) Filed: Apr. 28, 2023

Related U.S. Application Data
(60) Provisional application No. 63/336,490, filed on Apr. 29, 2022.

(51) Int. Cl. G09B 19/00; G06T 13/40; G06T 19/00; G02B 27/01

(52) U.S. Cl. CPC G09B 19/00; G06T 13/40; G06T 19/006; G02B 27/0101; G02B 27/017; G02B 2027/0138; G02B 2027/014

(57) ABSTRACT

A training simulator provides an immersive virtual training environment depicting an operator performing healthcare tasks to a patient. The simulator includes a head-mounted display unit (HMDU). The HMDU includes a camera, a speaker, a display, and a sensor providing visual and audio output to the operator. The simulator also includes one or more controllers, each having a sensor. The controller sensors and the HMDU sensor output signals representing spatial positioning, angular orientation and movement data of the controllers relative to the patient. The simulator includes a data processing system that models and renders the patient including a condition of the patient, the operator, used healthcare equipment and supplies, changes to the condition of the patient and the used healthcare equipment and supplies, and sensory guidance as to the operator's performance of the healthcare tasks to simulate the virtual training environment and to evaluate the operator's performance.

### CROSS-REFERENCE TO RELATED APPLICATIONS

This application claims benefit of and priority under 35 U.S.C. § 119(e) to copending, U.S. Patent Application Ser. No. 63/336,490, filed Apr. 29, 2022, the disclosure of which is hereby incorporated by reference herein in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the United States Patent and Trademark Office patent files or records, but otherwise reserves all copyright rights whatsoever.

### BACKGROUND OF THE INVENTION

### 1. Technical Field

The present invention relates generally to a training system employing computer simulation and immersive virtual reality for instructing and evaluating the progress of a person performing a skilled-oriented task and, more particularly, to a simulator for instructing and evaluating performance of a skilled-oriented task such as, for example, providing direct health care and/or assisting in providing care for a patient's needs, including perineal care, in a healthcare facility such as, for example, a residential facility, healthcare office, hospital or trauma center or facility, as well as on a scene of an event such as, for example, a motor vehicle accident, natural or manmade disaster, concert or other entertainment performance, and the like, or transportation therefrom to the healthcare facility, where care is provided under non-critical and/or critical conditions.

### 2. Related Art

Generally speaking, training is needed for a person to acquire and/or maintain skills necessary for performing a skill-oriented task such as, for example, providing and/or assisting patients in a healthcare facility address their direct health care needs. Health care needs include providing and/or assisting with perineal care to patients unable or unwilling to properly clean private areas such as, for example, genitals of both male and female patients, that can be particularly prone to infection.

In healthcare facilities patient care and safety are mission critical tasks. Many medical practitioners (e.g., doctors and nurses) undergo years of educational and practical (e.g., “on-the-job”) training to acquire, refine and/or maintain skills needed in the healthcare industry. The training necessary for these advance medical practitioners to acquire and/or maintain their skills applies also to other medical professionals such as, for example, emergency medical technicians (EMTs), licensed practical nurse (LPN), a certified nursing assistant also referred to as a nurse's aid or a patient care assistant (collectively referred to herein as a CNA), as well as individuals providing home health aid. These medical professionals typically work directly with a patient and/or with a nurse to assist in rendering patient care which can include many physical tasks of patient care. Tasks include, for example, bathing, grooming, and feeding patients, responding to patient's requests for assistance with positioning in bed, transport to restroom facilities, and the like, cleaning a patient as well as the patient's bedding and a patient's room or portion thereof, checking and restocking medical supplies located in proximity to the patient being cared for, taking or assisting other medical practitioners taking a patient's vital signs (e.g., temperature, blood pressure, and the like) or being administered medicine, and similar medical and patient care tasks. Often, an important part of each task performed includes documenting medical records so that other medical practitioners rendering care to a patient are fully informed of the patient's condition and what has been provided to the patient in a given time period.

Traditionally, these medical professionals (e.g., the EMTs, LPNs, CNAs, home health aid provider) acquire their skills initially in a classroom or other instructional setting, followed by working in a supervised, practical setting where some patient interaction occurs in a type of apprenticeship or “on-the-job” type training environment with another more experienced medical practitioner (e.g., a nurse or more experienced EMT, LPN, or CNA). As can be appreciated, there is a constant need for qualified and experience medical practitioners at all levels of patient care. Accordingly, there is a great demand for systems to assist in training medical practitioners.

Accordingly, there is a need for training systems and methods using computer simulation and immersive virtual reality and which permit evaluation of the progress in obtaining and/or maintaining skills needed by a medical practitioner such as, for example, EMTs, LPNs, CNAs, or home health aid providers, that assists patients in healthcare or residential facilities with their direct health care needs.

### SUMMARY OF THE INVENTION

The present invention is directed to a simulator for skill-oriented training of a task. The simulator includes a head-mounted display unit (HMDU) wearable by an operator operating the simulator. The HMDU has at least one of a camera, a speaker, a display device, and a HMDU sensor. The camera, the speaker, and the display device provide visual and audio output to the operator. The simulator also includes one or more controllers operable by the operator. The controllers each have at least one controller sensor. The controller sensor and the HMDU sensor cooperate to measure and to output one or more signals representing spatial positioning, angular orientation, speed and direction of movement data of the HMDU and/or the one or more controllers relative to a simulated patient as the operator performs a healthcare task. The simulator also includes a data processing system operatively coupled to the HMDU and the one or more controllers. The data processing system includes a processor and memory operatively coupled to the processor with a plurality of executable algorithms stored therein. The processor is configured by the executable algorithms to determine coordinates of a position, an orientation, and a speed and a direction of movement of the one or more controllers in relation to the patient as the operator takes actions to perform the healthcare task based on the one or more signals output from the HMDU sensor and the controller sensor of each of the one or more controllers. The processor is also configured to model the actions taken by the operator to perform the healthcare tasks to determine use of healthcare equipment and supplies and changes in condition of the patient and the used healthcare equipment and supplies in relation to the actions taken. The processor renders the patient, the used healthcare equipment and supplies, the condition of the patient, changes to the condition of the patient, changes to the used healthcare equipment and supplies, and sensory guidance as to the performance of the healthcare tasks from the actions taken by the operator in a three-dimensional virtual training environment. The processor is further configured to simulate in real-time the three-dimensional virtual training environment depicting the rendered patient, the rendered used healthcare equipment and supplies, the rendered changes to the condition of the patient, the rendered changes to the used healthcare equipment and supplies, and the rendered sensory guidance as the operator performs the healthcare task in the training environment.

In one embodiment, the rendered patient, the rendered used healthcare equipment and supplies, the rendered changes to the condition of the patient, the rendered changes to the used healthcare equipment and supplies, and the rendered sensory guidance are exhibited in near real-time to the operator within the training environment on the display device of the HMDU to provide in-process correction and reinforcement of preferred performance characteristics as the operator performs the healthcare task. In one embodiment, the rendered sensory guidance includes a plurality of visual, audio and tactile indications of performance by the operator as compared to optimal values for performance.

In one embodiment, the simulator further includes an avatar or portion thereof, manipulated and directed by the operator with the one or more controllers to take the actions to perform the healthcare task in the training environment. In one embodiment, the portion of the avatar includes virtual hands.

In one embodiment, the operator of the simulator further includes a plurality of operators undertaking the skill-oriented training as a group cooperating to perform the healthcare task within the three-dimensional virtual training environment. In still another embodiment, the operator is one of a medical professional and an individual providing home health aid. In one embodiment, the medical professional includes at least one of an emergency medical technician (EMT), a licensed practical nurse (LPN), and a certified nursing assistant, nurse's aid, or a patient care assistant referred to herein as a CNA.

In one embodiment, the sensory guidance exhibited to the operator and/or others includes one or more of visual, audio, and tactile indications of performance by the operator operating the one or more controllers relative to the patient as compared to optimal values for performance of the healthcare task or tasks currently being performed by the operator. In one embodiment, the visual indications of performance include an indication, instruction, and/or guidance of the optimal values for preferred performance of the healthcare task currently being performed by the operator. In one embodiment, the audio indications of performance include an audio tone output by the at least one speaker of the HMDU. In still one embodiment, the audio tone is a reaction by the patient to the healthcare task or tasks currently being performed by the operator.

In yet another embodiment, the simulator further includes a display device operatively coupled to the data processing system such that an instructor may monitor the performance by the operator of the healthcare task. In one embodiment, the visual indications include a score or grade established by the instructor for the operator in the performance by the operator of the healthcare task as compared to a set of performance criteria defining standards of acceptability. In one embodiment, the established score or grade is a numeric value based on how close to optimum the operator's performance is to the set of performance criteria. In one embodiment, the score or grade further includes rewards including certification levels and achievements highlighting the operator's results and/or progress as compared to the set of performance criteria and to other operators. In still another embodiment, the score or grade and rewards for one or more of the operators are at least one of shared electronically, posted on a website or bulletin board, and over social media sites.

In one embodiment of the simulator, the data processing system is configured to provide a review mode for evaluating the operator's performance of the healthcare task. In one embodiment, when in the review mode the data processing system is further configured to provide reports of the operator's performance. In one embodiment, the data processing system is further configured to provide the review mode to at least one of the operator of the controller, an instructor overseeing the skill-oriented training, and other operators undergoing the skill-oriented training. In one embodiment, the simulator is portable as a self-contained modular assembly. In one embodiment, the data processing system of the simulator is further configured to provide one or more modes for assigning characteristics of at least one of the operator, the patient, and the environmental setting where the healthcare task is performed.

### BRIEF DESCRIPTION OF THE DRAWINGS

Referring now to the Figures, which are exemplary embodiments, and wherein the like elements are numbered alike.

FIG. 1 is a schematic diagram of a healthcare training simulator defining and operating within a three-dimensional healthcare training environment, according to one embodiment of the present invention.

FIG. 2A depicts a head-mounted display unit utilized in the training simulator of FIG. 1, according to one embodiment of the present invention.

FIG. 2B depicts a controller utilized in the training simulator of FIG. 1, according to one embodiment of the present invention.

FIG. 3 is a simplified block diagram of components of the training simulator of FIG. 1, according to one embodiment of the present invention.

FIG. 4A is a graphical user interface depicting an exemplary sign-in page where a user enters his/her credentials as an authorized user of the training simulator of FIG. 1, according to one embodiment of the present invention.

FIG. 4B is a graphical user interface depicting an exemplary start-up page where a user invokes the training simulator of FIG. 1 to monitor his/her performance of care within a 3-D virtual healthcare training environment, according to one embodiment of the present invention.

FIGS. 5A to 5D are graphical user interfaces depicting an operator and/or operators using the training simulator of FIG. 1 to identify and verify/confirm a resident/patient that is scheduled to receive healthcare within the 3-D virtual healthcare training environment, according to one embodiment of the present invention.

FIGS. 6A to 6M are graphical user interfaces depicting an operator and/or operators using the training simulator of FIG. 1 to perform a healthcare task within the 3-D virtual healthcare training environment, according to one embodiment of the present invention.

FIGS. 7A to 7E are graphical user interfaces depicting the operator and/or operators using the training simulator of FIG. 1 to perform another healthcare task within the 3-D virtual healthcare training environment, according to one embodiment of the present invention.

FIGS. 8A to 8D are graphical user interfaces depicting the operator and/or operators using the training simulator of FIG. 1 to perform and/or setup sensors to perform another healthcare task within the 3-D virtual healthcare training environment, according to one embodiment of the present invention.

FIGS. 9A to 9D are graphical user interfaces depicting the operator and/or operators responding to the training simulator of FIG. 1 in an exemplary test or quiz to evaluate performance of a healthcare task within the 3-D virtual healthcare training environment, according to one embodiment of the present invention.

FIGS. 10A and 10B are graphical user interfaces depicting the operator and/or operators using the training simulator of FIG. 1 to perform another healthcare task within the 3-D virtual healthcare training environment, according to one embodiment of the present invention.

FIG. 11 is graphical user interfaces depicting the operator and/or operators using the training simulator of FIG. 1 to perform another healthcare task within the 3-D virtual healthcare training environment, according to one embodiment of the present invention.

FIG. 12 is graphical user interfaces depicting the operator and/or operators using the training simulator of FIG. 1 to perform another healthcare task within the 3-D virtual healthcare training environment, according to one embodiment of the present invention.

FIGS. 13A to 13F are graphical user interfaces depicting reports provided by a reporting feature of the training simulator of FIG. 1, according to one embodiment of the present invention.

FIGS. 14A to 14D are graphical user interfaces depicting a customization of a resident/patient's condition and depicting the operator and/or operators using the training simulator of FIG. 1 to perform a healthcare task based on the resident/patient's condition within the 3-D virtual healthcare training environment, according to one embodiment of the present invention.

FIGS. 15A to 15D depict a portability feature of the training simulator of FIG. 1, according to one embodiment of the present invention.

FIGS. 16A to 16G depict customization features of the training simulator of FIG. 1, according to one embodiment of the present invention.

### DESCRIPTION OF THE INVENTION

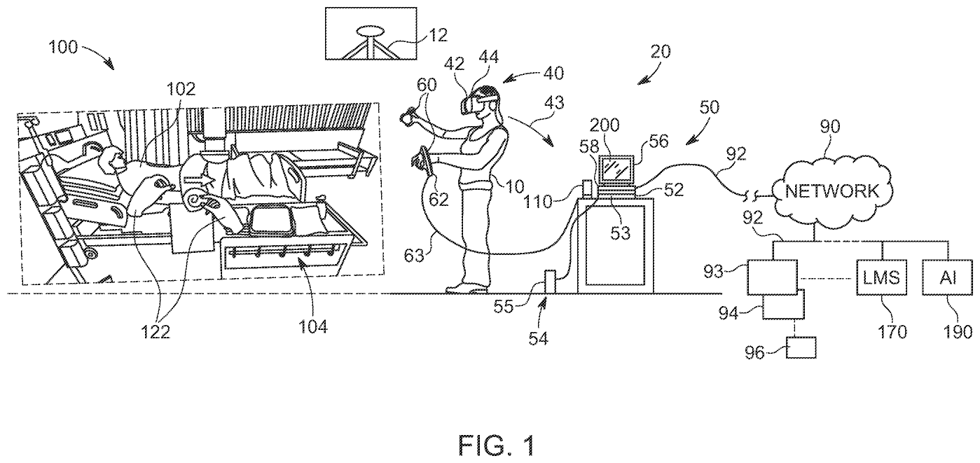

FIG. 1 depicts an operator 10 operating a VRNA™ healthcare training simulator 20 to train, for example, to develop and/or improve his/her skills in performing a skill-oriented task and/or steps thereof such as, for example, providing health care and/or assisting a resident or a patient 102 in a virtual healthcare environment 100 attend to his/her direct health care needs. VRNA is a trademark of VRSim, Inc. (East Hartford, CT USA). In one exemplary embodiment, health care includes providing and/or assisting residents or patients address perineal care. It should be appreciated that while the VRNA healthcare training simulator 20, as described herein, is utilized for instructing and evaluating performance of skilled-oriented tasks such as, for example, providing and/or assisting in providing healthcare for a patient's needs, including perineal care, in specific environments of a healthcare facility such as, for example, a residential facility, healthcare office, hospital or trauma center or facility, as well as on the scene of an event, such as a motor vehicle accident, natural or manmade disaster, concert or other entertainment performance, and the like, or transportation therefrom, where care is provided under non-critical and/or critical conditions, the disclosure merely provides exemplary uses and/or training environments, and is not intended to limit the scope of the present invention. It should also be appreciated that the terms resident and patient are used interchangeably in this disclosure to refer to persons receiving healthcare. The VRNA training simulator 20 provides for an evaluation of the skills demonstrated by the operator 10 in performing the skill-oriented task and steps thereof. The skills of the operator 10 include, for example, proper technique in performing and/or in assisting in the performance of the task, namely, his/her positioning and movement in rendering care consistently and in a preferred manner to promote the health and ensure the safety of the patient, operator, and others in proximity to the patient undergoing care. The skills of the operator 10 also include, for example, the proper use of and/or reading of measurements taken with medical tools and/or equipment. In one embodiment, tasks and steps thereof include, for example, bathing, grooming, and feeding patients, responding to patient's requests for assistance with positioning in his/her bed, periodic rotation for bedridden patients, transport to restroom facilities, and the like, cleaning bedding and a patient's room or portion thereof, checking and restocking medical supplies located in proximity to the patient being cared for, taking or assisting other medical practitioners taking a patient's vital signs (e.g., temperature, blood pressure, and the like) or being administered medicine, and similar medical and patient care tasks. Other tasks, steps, and skills are described throughout this disclosure. Often, an important part of each task performed includes documenting medical records so that other medical practitioners rendering care to a patient are fully informed of the patient's condition and what has been provided to the patient in a given time period. Tasks may also include using and, at times restocking, health care equipment and/or supplies including cleaning solutions, towels, gloves, masks, and other personal protective equipment (PPE). The skills evaluated during performance of such task may include not only how the physical task is performed but also other measures such as, for example, ensuring privacy for the patient 102 undergoing care as a patient's private areas, e.g., genitals, are exposed while care is being rendered. Additionally, tasks and skills include ensuring for the proper hygiene of not only the patient 102 but also of the care practitioner (e.g., operator 10), as both typically can be exposed to contamination during certain healthcare procedures. As described herein, the simulator 20 provides evaluation of such skills in real-time, e.g., as the task or steps of a healthcare procedure is being performed, and after one or more performances, e.g., in one or more review modes as described herein.

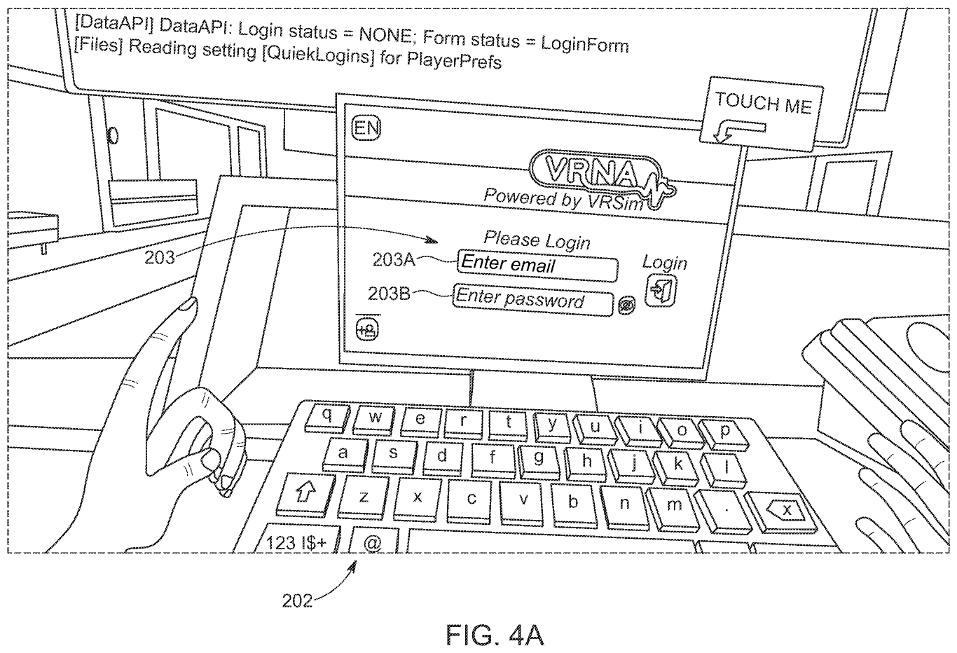

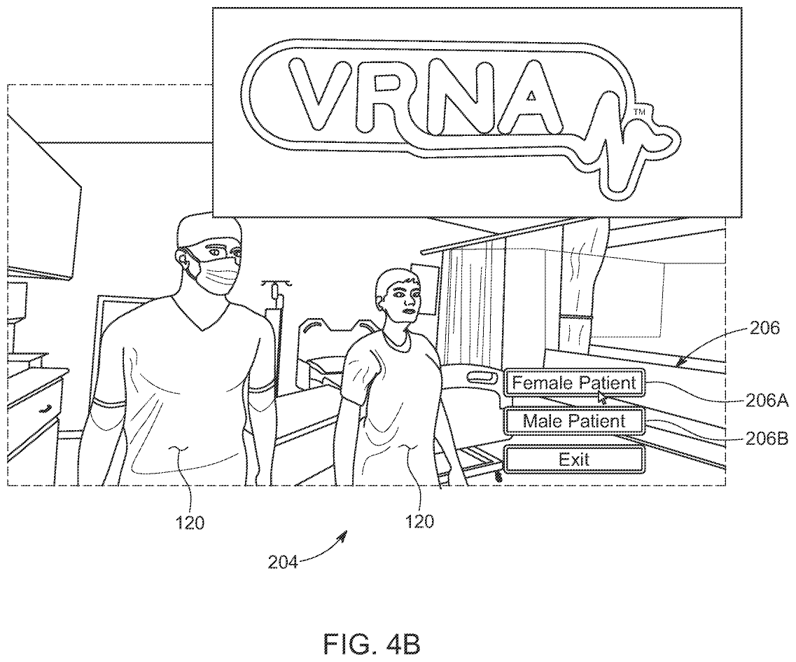

In one embodiment, the simulator 20 permits training and evaluating the operator's performance of a task, namely, using one or more controllers 60, for example, one or more handheld controllers 60 (e.g., a righthand and lefthand controller), to take actions by manipulating and directing a position and movement of an avatar 120 (FIG. 4), or portions thereof such as virtual hands 122 (FIG. 1), rendered in the virtual healthcare environment 100 during the performance of a healthcare procedure such as, for example, providing and/or assisting the patient 102 with his/her perineal care. As one skilled in the art appreciates, the avatar 120 is a graphical representation of the operator or user, or an operator/user-defined alter ego or character, employed within the virtual healthcare environment 100. In one embodiment, the operator or user may selectively vary characteristics of his/her avatar 120 including, for example, physical features such as gender, hair, skin tone, skin color, height, weight, and the like, clothing and/or accessories of a male or female healthcare provider, footwear, gloves, masks, and other personal protective equipment (e.g., equipment worn by the operator to minimize exposure to hazards that may cause workplace injuries and illnesses, collectively PPE). In one embodiment, an administer of the simulator 20, an instructor or certification agent 12 (described below), and/or the operator or user may selectively vary characteristics or physical features of the simulated resident or patient 102 such as gender, hair, skin tone, skin color, height, weight, and the like, clothing or medical gown worn by the patient 102, medical condition including mental and/or physical conditions, symptoms and/or disabilities of the resident or patient 102 such as, for example, height, weight, patients having an amputated limb or limbs, physical deformities, injuries, wounds, or other medical illnesses, diseases, handicaps, and/or special health care needs, and the like.

In one embodiment, the one or more handheld controllers 60 include a Pico Neo 3 controller of Qingdao Pico Technology Co., Ltd. dba Pico Immersive Pte. Ltd (Qingdao, China) (Pico Neo is a registered trademark of Qingdao Pico Technology Co., Ltd.). In one embodiment, the one or more handheld controllers 60 include an Oculus Quest 2 and/or an Oculus Rift controller of Facebook Technologies, LLC (Menlo Park, California) (Oculus Quest and Oculus Rift are registered trademarks of Facebook Technologies, LLC). In another embodiment, the one or more handheld controllers 60 include a Vive Pro Series controller of HTC Corporation (Taoyuan City Taiwan) (Vive is a registered trademark of HTC Corporation). In still another embodiment, it is within the scope of the present invention for the simulator 20 to be implemented in a controller-free embodiment, for example, where a user's hands and gestures made therewith (e.g., grasping, picking up and moving objects, pinching, swiping, and the like) are identified and tracked (e.g., with cameras and sensors within the virtual healthcare environment 100) rather than actions and movement initiated by the user with a handheld controller in the environment 100.

As described herein, the operator 10 using the one or more controllers 60 alone or with one or more other input devices 53 (described below) manipulates and directs the avatar 120 to navigate through the virtual healthcare environment 100 and to take actions, for example, with the virtual hands 122, objects 104 (e.g., the health care tools, equipment, PPE, and/or supplies) rendered therein, to perform tasks within the virtual healthcare environment 100. A tracking system within each of the one or more controllers 60 spatial senses and tracks movement of the respective controller 60 (e.g., speed, direction, orientation, spatial location, and the like) as directed by the operator 10 in performing one or more tasks in providing and/or assisting the resident or patient 102 with his/her healthcare needs, for example, perineal care needs. The healthcare training simulator 20 collects, determines and/or stores data and information (described below) defining the movement of the one or more controllers 60 including its speed, direction, orientation, and the like, as well as the impact of such movement and actions within the virtual healthcare environment 100 such as, for example, as health care equipment and/or supplies 104 are used and the condition of the patient 102 changes (e.g., improves) as the operator 10 renders care to the patient 102.

Referring to FIGS. 1, 2A, 2B, and 3, one or more video cameras 42 and sensors 44 (e.g., tracking sensors), and one or more display devices 46 provided on, for example, a head-mounted display unit (HMDU) 40 worn by the operator 10, cooperates with the one or more controllers 60 and sensors 62 (e.g., tracking sensors) thereof, to provide data and information to a processing system 50. From such data and information, the processing system 50 constructs a position (e.g., spatial location), orientation, and speed and/or direction of movement of the HMDU and/or the one or more controllers 60 in relation to the simulated patient 102, as the operator manipulates and directs the avatar 120 and/or the virtual hands 122 to take actions in performing the healthcare tasks rendered in the virtual healthcare environment 100. As each of the one or more controllers 60 is operated by the operator 10, the processing system 50 executes algorithms (e.g., one or more algorithms or subsystems 132 described below) to determine coordinates of, for example, a position (e.g., spatial location), orientation, and movement, the HMDU 40 and/or the controller 60 in relation to the simulated patient 102. The processing system 50 executes the algorithms to model the actions directed by the operator 10 in performing healthcare tasks to the simulated patient 102, his/her use of objects 104 in the performance of such tasks, and changes to the condition of the simulated patient 102 and/or objects 104 within the virtual healthcare environment 100. The processing system 50 executes the algorithms to render the avatar 120 and/or the virtual hands 122, the simulated patient 102, the objects 104, the condition of the patient 102, a reaction of the simulated patient 102 (e.g., groan, vocal outburst, movement, and the like), and/or actions taken in a three-dimensional (3-D) virtual healthcare training environment 100 in response to the modeled performance of the healthcare tasks, and to simulate, in real-time, the 3-D virtual healthcare training environment 100 depicting the rendered the avatar 120 and/or the virtual hands 122, the simulated patient 102, the objects 104, the condition of the patient, changes to the condition and/or reaction of the simulated patient 102 and/or the objects 104 used, and the actions taken with virtual imagery as the operator 10 performs the healthcare tasks.

As should be appreciated, the objects 104 within the 3-D virtual VRNA healthcare training environment 100 include, for example, health care tools and/or equipment, PPEs, and/or supplies. It should also be appreciated that the 3-D virtual healthcare training environment 100 not only depicts the simulated patient 102 but also a condition of and/or symptoms and/or reaction exhibited by the simulated patient 102 undergoing treatment, including for example, changes in conditions, symptoms, and/or reactions of the patient 102 before, during, and after care. In one embodiment, the depicted condition and/or symptoms of simulated patient 102 are related to perineal care and may include, for example, affects from episodes of incontinence, bedsores, skin ulcers, or the like. The operator 10 interacts within the virtual reality provided in the 3-D virtual healthcare training environment 100, for example, to view and otherwise sense (e.g., see, feel, hear, and optionally smell) the patient 102 and/or their condition, the avatar 120 and/or virtual hands 122, and the resulting actions he/she is directing to the simulated patient 102, their condition and changes thereto, and the objects 104 used (e.g., health care tools, equipment, PPEs, and/or supplies) and changes thereto, as he/she performs the healthcare tasks. In one embodiment, multiple operators 10 are present simultaneously within the 3-D virtual healthcare training environment 100 and cooperate to provide and assist in providing healthcare to the patient 102. The interaction (individual operator and/or group of operators) is monitored, and data and information therefrom is recorded and stored (e.g., in a memory device) to permit performance evaluation by the operator 10, an instructor or certification agent 12, and/or other operators/healthcare trainees present during training or otherwise monitoring or cooperating to provide healthcare within the 3-D virtual healthcare training environment 100 at or from another location remote from where the training is being conducted, as is described in further detail below.

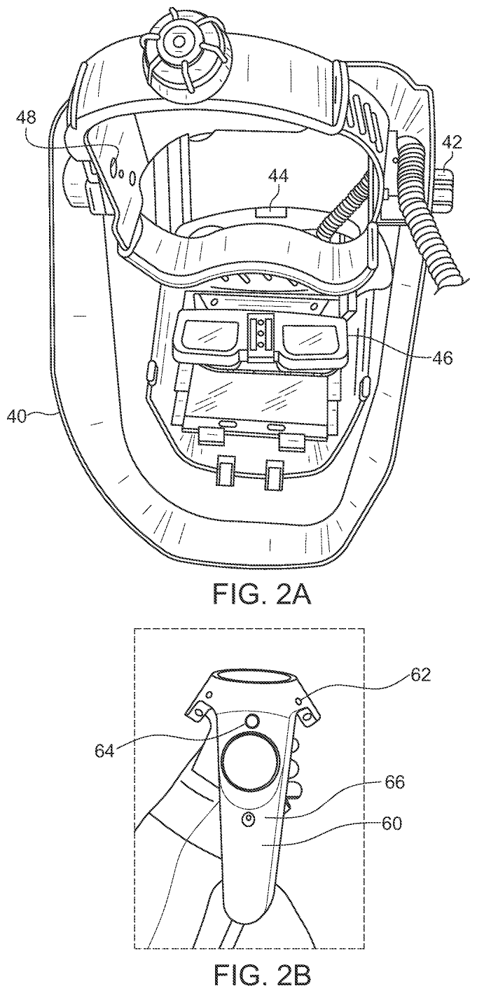

In one embodiment, the healthcare training simulator 20 generates audio, visual, and other forms of sensory output, for example, vibration, workplace disturbance (e.g., noise, smells, interruption from other medical practitioners and/or patient visitors, etc.), environmental conditions (e.g., lighting) and the like, to simulate senses experienced by the operator 10, individually and as a group of operators, as if the healthcare procedure is being performed in a real-world healthcare setting. For example, the training simulator 20 simulates experiences that the operator 10 (individual) and/or operators 10 (group) may encounter when performing the healthcare task “in the field,” e.g., outside of the training environment and in a healthcare work environment. As shown in FIG. 2A, the HMDU 40 includes one or more display devices 46 and one or more audio speakers 48 that provide images and sounds generated by the healthcare training simulator 20 to the operator 10. In keeping with a goal of accurately simulating real-world settings and work experiences within the 3-D virtual healthcare training environment 100, the simulator 20 emulates characteristics of an actual healthcare environment and/or treatment facility including, for example, the sound, disturbances, and environmental conditions the operator 10 may experience while performing healthcare task to a patient. For example, and as illustrated in FIG. 1, the 3-D virtual healthcare training environment 100 depicts health care equipment and/or supplies utilized in rendering care. In one embodiment, the training simulator 20 may depict other patients, healthcare providers (simulated or actively participating as operators), or visitors in proximity to the simulated patient 102 undergoing care from the operator 10 to evaluate actions the operator 10 takes to maintain his/her composure and concentration when rendering care (individually or as a member of a group) as well as providing privacy to the patient 102. For example, a determination may be made as to whether the operator 10 closed curtains or other barriers to prevent, or at least restrict, third parties from viewing private areas of the simulated patient 102.

In one embodiment, input and output devices of the HMDU 40 and each of the one or more controllers 60 such as, for example, the cameras 42, the sensors 44 (e.g., tracking sensors), the display 46, and the speakers 48 of the HMDU 40, and sensors 62 (e.g., tracking sensors), control buttons or triggers 64, and haptic devices 66 of the controller 60 (e.g., rumble packs to simulate weight and/or vibration) that impart forces, vibrations and/or motion to the operator 10 of the controllers 60, and external input and output devices such as speakers 55, are incorporated into the conventional form factors. Signals from these input and output devices (as described below) are input signals and provide data to the processing system 50. The data is processed and provided to permit a thorough evaluation of the healthcare training procedure including the actions taken by the operator 10 in performing healthcare and equipment and/or supplies used therein.

As should be appreciated, the HMDU 40 and the one or more controllers 60 provide a plurality of inputs to the healthcare training simulator 20. The plurality of inputs includes, for example, spatial positioning (e.g., proximity or distance), orientation (e.g., angular relationship), and movement (e.g., direction and/or speed) data and information for tracking the position of one or more of the HMDU 40 and the one or more controllers 60 relative to the simulated patient 102, objects 104 (e.g., healthcare tools, equipment, PPEs, and supplies) within the 3-D virtual healthcare training environment 100. The HMDU 40 and the one or more controllers 60 may include sensors (e.g., the tracking sensors 44 and 62) that track the movement of the operator 10 operating the controllers 60. In one embodiment, the sensors 44 and 62 may include, for example, magnetic sensors, mounted to and/or within the HMDU 40 and the controllers 60 for measuring spatial position, angular orientation, and movement within the 3-D virtual healthcare training environment 100. In one embodiment, the sensors 44 and 62 of the HMDU 40 and the controllers 60 are components of a six degree of freedom (e.g., x, y, z for linear direction, and pitch, yaw, and roll for angular direction) tracking system 110. In one embodiment, the tracking system is an “inside-out” positional tracking system, where one or more cameras and/or sensors are located on the device being tracked (e.g. the HMDU 40 and controllers 60) and the device “looks out” to determine how its spatial positioning, orientation, and movement has changed in relation to the external environment to reflect changes (e.g., in spatial positioning, orientation, and movement) within the 3-D virtual healthcare training environment 100. Examples of systems employing such inside-out positional tracking include, for example, the aforementioned Oculus Quest, Oculus Rift, and Vive controllers and HMDUs. In another embodiment, the tracking system is an “outside-in” positional tracking system, where one or more cameras and/or sensors are fixedly located in the environment (e.g., including one or more stationary locations) and on the device being tracked (e.g. the HMDU 40 and controllers 60) and the spatial positioning, orientation, and movement of the device being tracked is determined in relation to the stationary locations within the 3-D virtual healthcare training environment 100. An example of a system employing such outside-in positional tracking includes, for example, a Polhemus PATRIOT™ Tracking System, model number 4A0520-01, from the Polhemus company (Colchester, Vermont USA).

It should be appreciated that it is within the scope of the present invention to employ other tracking systems for locating the HMDU 40 and/or the controllers 60 in relation to the patient 102 within the 3-D virtual VRNA healthcare training environment 100. For example, in some embodiments the training simulator 20 includes a capability to automatically sense dynamic spatial properties (e.g., positions, orientations, and movements) of the HMDU 40 and/or the controllers 60 during performance of one or more tasks in providing and/or assisting in the performance of the task, namely, his/her positioning and movement in rendering care consistently and in a preferred manner. The training simulator 20 further includes the capability to automatically track the sensed dynamic spatial properties of the HMDU 40 and/or one or more of the controllers 60 over time and automatically capture (e.g., electronically capture) the tracked dynamic spatial properties thereof during the performance of the healthcare tasks.

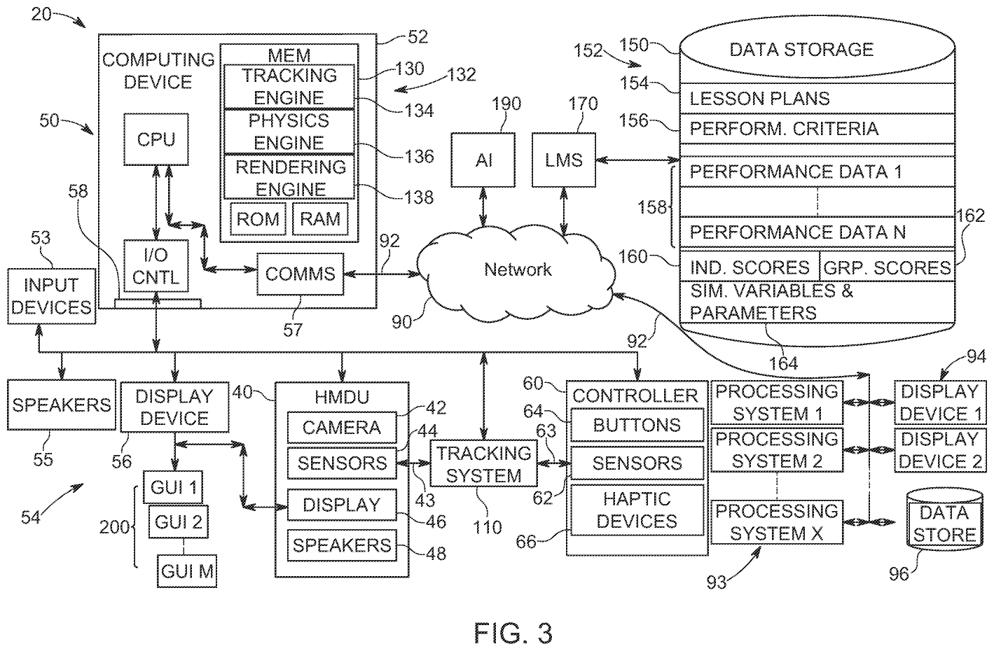

As shown in FIGS. 1 and 3, the sensors 44 and 62 output data that is received by the tracking system 110 over a wired and/or wireless communication connections 43 and 63 (e.g., provide input) and provided to the processing device 50 for use in determining the operator's 10, the HMDU's 40, and the one or more controllers' 60 movement within the 3-D VRNA healthcare training environment 100, e.g., in relation to the simulated patient 102 and the other objects 104 (e.g., the health care equipment and/or supplies) in the environment 100.

In one embodiment, as illustrated in FIG. 3, a simplified block diagram view of the healthcare training simulator 20, the processing system 50 is a standalone or networked computing device 52 having or operatively coupled to one or more microprocessors (CPU), memory (e.g., internal memory 130 including hard drives, ROM, RAM, and the like), and/or data storage devices 150 (e.g., hard drives, optical storage devices, and the like) as is known in the art. The computing device 52 includes one or more input devices 53 such as, for example, a keyboard, mouse or like pointing device, touch screen portions of a display device, ports 58 for receiving data such as, for example, a plug or terminal receiving the wired communication connections 43 and 63 from the sensors 44 and 62 directly or from the tracking system 110, and one or more output devices 54. The output devices 54 include, for example, one or more display devices operative coupled to the computing device 52 to exhibit visual output, such as, for example, the one or more display devices 46 of the HMDU 40 and/or a monitor 56 coupled directly to the computing device 52 or a portable computing processing system (e.g., processing systems 93, described below) such as, for example, a personal digital assistant (PDA), IPAD, tablet, mobile radio telephone, smartphone (e.g., Apple™ iPhone™ device, Google™ Android™ device, etc.), or the like. The one or more output devices 54 also include, for example, one or more speakers 55 operative coupled to the computing device 52 to produce sound for auditory perception by the operator 10 and others. In one embodiment, the output devices 54 exhibit one or more graphical user interfaces (GUIs) 200 (as described below) that may be visually perceived by the operator 10 operating the coating simulator the instructor or certification agent 12, and/or other interested persons such as, for example, other medical trainees, observing and evaluating the operator's 10 performance.

In one embodiment, illustrated in FIGS. 1 and 3, the processing system 50 includes network communication circuitry (COMMS) 57 for operatively coupling the processing system by wired or wireless communication connections 92 to a network 90 such as, for example, an intranet, extranet, or the Internet, and to a plurality of processing systems 93, display devices 94, and/or data storage devices 96. In one embodiment, described in detail below, the communication connection 92 and the network 90 provide an ability to share performance and ratings (e.g., scores, rewards and the like) between and among a plurality of operators (e.g., classes or teams of students/healthcare trainees) via such mechanisms as electronic mail, electronic bulletin boards, social networking sites, a Performance Portal™ website (described below), and the like, for example, via the one or more GUIs 200. Performance Portal is a trademark of VRSim, Inc. (East Hartford, CT USA). In one embodiment, as also described in detail below, the communication connection 92 and the network 90 provide connectivity and operatively couple the VRNA healthcare training simulator 20 to a Learning Management System (LMS) 170.

In one embodiment, the computing device 52 of the processing system 50 invokes one or more algorithms or subsystems 132 that are stored in the internal memory 130 or hosted at a remote location such as, for example, a processing device (e.g., one of the processing systems 93) or in one of the data storage devices 96 or 150 operatively coupled to the computing device 52. From data and information provided by the HMDU 40 and one or more controllers 60, the one or more algorithms or subsystems 132 are executed by the CPU of computing device 52 to direct the computing device 52 to determine coordinates of a position, an orientation, and a speed and direction of movement of the operator 10 (e.g., via data and information received from the sensors 44 and 62 of the HMDU 40 and/or controllers 60) to model, render, and simulate the 3-D virtual training environment 100 depicting the rendered the avatar 120 and/or the virtual hands 122, patient 102 and/or the other objects 104 (e.g., the health care tools, equipment and/or supplies) with virtual imagery as the operator 10 performs the healthcare tasks.

In one embodiment, the algorithms or subsystems 132 include, for example, a tracking engine 134, a physics engine 136, and a rendering engine 138. The tracking engine 134 receives input, e.g., data and information, from the healthcare training environment 100 such as a spatial position (e.g., proximity and distance), and/or an angular orientation, as well as a direction, path and/or speed of movement of the sensors 44 and 62 of the HMDU 40 and/or the one or more controllers 60, respectively, in relation to the patient 102 and the objects 104 in the training environment 100 as provided by the sensors 44 and 62 of the HMDU 40 and/or each of the one or more controllers 60. The tracking engine 134 processes the input and provides coordinates to the physics engine 136. The physics engine 136 models the actions directed by the operator and/or operators 10 in performing healthcare tasks to the patient 102, the use of the objects 104 (e.g., the health care tools, equipment and/or supplies) in the performance of such tasks, and changes to the condition of the patient and to the used healthcare equipment and supplies within the virtual healthcare environment 100 based on the received input and/or coordinates from the tracking engine 134. The physics engine 136 provides the modeled actions performed by the operator or and/or operators 10 to the rendering engine 138. The processing system 50 then executes the algorithms of the rendering engine 138 to render the avatar 120 and/or the virtual hands 122 for the operator and/or operators 10, the patient 102, the patient's condition, the use of the objects 104 (e.g., the health care tools, equipment and/or supplies) in the performance of such tasks, and changes to the condition of the patient and to the used healthcare equipment and supplies in a three-dimensional (3-D) virtual healthcare training environment 100 in response to the modeled performance of the healthcare tasks. The processing system 50 then simulates, in real-time, the 3-D virtual healthcare training environment 100 depicting the rendered the avatar 120 and/or the virtual hands 122 of the operator and/or operators 10, the simulated patient 102, the used objects 104, the changes to the condition and/or reaction of the patient and/or the used healthcare equipment and supplies with virtual imagery as the operator and/or operators 10 perform the healthcare tasks.

In one embodiment, the operating environment of the VRNA healthcare training simulator 20 is developed using a Unity™ game engine (Unity Technologies, San Francisco, California USA; and Unity IPR ApS, Copenhagen, DENMARK) and operates on the Windows™ (Microsoft Corporation, Redmond, Washington USA) platform. It should be appreciated, that the VRNA healthcare training simulator 20 may also operate on a portable computing processing system, for example, the aforementioned processing systems 93 including PDAs, IPADs, tablet computers, mobile radio telephones, smartphones (e.g., Apple™ iPhone™ device, Google™ Android™ device, etc.), or the like. It should be appreciated that one or more of the algorithms or subsystems 132 described herein (e.g., the tracking engine 134, the physics engine 136, and the rendering engine 138) may access the data storage device 150 to retrieve and/or store data and information 152 including data and information describing training and/or lesson plans 154 including skilled-oriented tasks, steps, or activities in providing care and/or in assisting patients with direct healthcare needs, performance criteria 156 (e.g., proper techniques for performing and/or assisting in performing a healthcare task), data and information from one or more instances of performance of healthcare tasks 158 by one or more healthcare trainees (e.g., operators 10), scores and/or performance evaluation data for individual 160 and/or groups 162 of healthcare trainees (e.g., one or more healthcare trainees/operators 10), and healthcare simulation data as well as variables and/or parameters 164 used by the healthcare training simulator 20. It should be appreciated that the input data and information is processed by the computing device 52 in near real-time such that the position, distance, orientation, path, direction, and speed of movement of the HMDU 40 and/or one or more controllers 60 is depicted as the operator and/or operators 10 are performing one or more healthcare tasks. Further aspects of the training simulator 20, are described in detail below.

It also should be appreciated that the input data and information include one or more variables or parameters set by the operator 10 on healthcare tools or equipment such as, for example, one or more setting for medical devices that measure, as is known in the art, temperature, blood pressure, or the like, of the patient 102 undergoing care. Moreover, the operator 10 may enter parameters, measurements, tasks performed, condition of a patient as observed by the operator 10 and the like, in electronic medical records to simulate the documenting of care administered to the patient 102 as the operator 10 performs healthcare tasks within the 3-D virtual training environment 100. In effect, the tracking engine 134, the physics engine 136, and the rendering engine 138 simulate actions taken by the operator and/or operators 10 in performing healthcare tasks in a non-virtual environment. In one embodiment, the actions taken by the operator and/or operators 10 in performing healthcare tasks are evaluated and compared to preferred and/or proper techniques for performing and/or assisting in performing healthcare tasks (e.g., performance criteria 156). The actions of the operator and/or operators 10 can then be viewed in, for example, in one or more review or evaluation modes, a specific instructional mode, and/or a playback mode, where the actions of the operator 10 are shown to the operator 10 (e.g., the healthcare trainee or trainees), the instructor or certification agent 12, and/or other healthcare trainees.

For example, the actions of the operator and/or operators 10, and for example, the acceptability thereof in performing healthcare tasks with preferred and/or proper technique, reflect the level of skill of the operator and/or operators 10 individually and as a group. As can be appreciated, good technique typically results in acceptable actions in performing healthcare tasks, and less than good technique may result in an unacceptable action in performing healthcare tasks. The evaluation, and various review modes thereof (described herein), allows the operator 10, an instructor or certification agent 12 and/or others (e.g., other healthcare trainees) to evaluate the technique and actions used in performing healthcare tasks in a virtual setting, as captured and stored by the training simulator 20, for example, as performance data 158, and to make in-process adjustments to or to maintain the preferred or proper technique being performed and/or performed in a next healthcare performance. The evaluation compares the demonstrated techniques to acceptable performance criteria for the task (e.g., the performance criteria 156) and ultimately the acceptability of the tasks performed by the operator and/or operators 10 to the patient 102. In one embodiment, the operator's performance as he/she completes one or more skilled-oriented tasks, steps, or activities in providing care and/or in assisting patients with direct healthcare needs (e.g., within the training and/or lesson plans 154) is monitored and graded, scored or otherwise evaluated in comparison to preferred or proper techniques for performing and/or assisting in performing the healthcare task (e.g., in accordance with the performance criteria 156). The grade, score and/or other evaluation information (e.g., comments from the instructor 12), operator's progress in obtaining requisite level of knowledge or skill in a task or tasks, may be stored in the data storage device 150 as, for example, scores and/or performance evaluation data for an individual 160 and/or for one or more groups 162 of healthcare trainees. In one embodiment, the review modes may be utilized to evaluate an operator's knowledge of acceptable and/or unacceptable aspects of a previous performance by the operator and/or operators 10 or by an actual or theoretical third-party operator. For example, a review mode may present a number of deficiencies in a performance of one or more healthcare tasks and query the operator 10 to identify the type or nature of the deficiency in the performance, possible reasons for the deficiency, and/or how to correct the deficiency going forward or in remedial operations.

It should be appreciated that it is also within the scope of the present invention for the review modes to provide tutorials, e.g., audio-video examples, illustrating setup and use of healthcare equipment and supplies typically used in the healthcare industry, acceptable performance techniques using the same, common deficiencies and ways to reduce or eliminate the same, and the like. It should also be appreciated that, as described herein, the VRNA healthcare training simulator 20 can be used for training, developing, maintaining, and improving other skills (e.g., more than just performance of healthcare treatment procedures) but also skills such as, for example, workplace safety, patient privacy, team building, and group performance skills, and the like.

It should further be appreciated that the VRNA healthcare training simulator 20 may be implemented as a project-based system wherein an individual instructor, certification agent, or the like, may define their own performance characteristics (e.g., elapsed time, preferred and/or proper performance techniques, requisite level of knowledge or skill to attain a rating or certification, and the like) and/or criteria including those unique to the instructor, agent and/or a given healthcare facility. In such embodiments, the operator and/or operators 10 are evaluated (e.g., individually and as a group) in accordance with the unique performance characteristics and/or criteria. In one embodiment, as described herein, the healthcare training simulator 20 is operatively coupled to the Learning Management System (LMS) 170. The LMS 170 may access the data storage device 150 that stores data and information 152 used by the healthcare training simulator 20.

In one embodiment, the healthcare training simulator 20 is operatively coupled to an Artificial Intelligence (AI) engine 190. The AI engine 190 is operatively coupled, directly or through the network 90, to the computing device 50 and/or the LMS 170. In one embodiment, the AI engine 190 accesses and analyzes data and information 152 within the LMS 170 and/or data storage device 150 including the performance criteria 156, the performance data 158, scores and/or evaluation data for individual 160 and/or groups 162, and the like, for one or more of the operators 10 and identifies, for example, successes or deficiencies in performance by individual and/or groups of operators 10, successes or deficiencies or instructors in terms of how his/her trainees performed, and the like. In one embodiment, the AI engine 190 determines common and/or trends in deficiencies and recommends modifications to existing and/or new lesson plans, tasks, and activities (e.g., the stored lesson plans 154), and/or to the performance criteria 156, with an aim of minimizing and/or substantially eliminating the identified and/or determined deficiencies through performance of the improved and/or new lesson plans and evaluation thereof by improved and/or new performance criteria 156. It should be appreciated that the AI engine 190 may access and analyze performance data on-demand or iteratively to provide continuous learning improvements over predetermined and/or prolonged periods. In one embodiment, the AI engine 190 interacts with the operator and/or operators 10 (e.g., respective avatars), for example, as an in-scene instructor (e.g., senior medical practitioner), or to provide and/or to enhance interaction to be more realistic of actual conditions in a healthcare facility or in an interior or exterior scene of an event (e.g., motor vehicle accident, critical natural or manmade disaster, concert or other entertainment performance, and the like) under ordinary daily and/or emergency conditions. It should be appreciated that the scene of the event and simulated patient interaction may include in-transport care as the patient is being moved, e.g., driven or flown, from an accident sight to a hospital or other trauma center.

FIGS. 4A and 4B depict two of the GUIs 200 exhibiting an exemplary sign-in page 202 and start-up page 204 where a user (e.g., operator 10) invokes the training simulator 20. As should be appreciated, the GUIs 200, such as the sign-in page GUI 202 and the start-up page GUI 204, are presented by the data processing system 50 on one or more of the display devices 56 and 94 coupled to the computing device 52 and/or the display 46 of the HMDU 40. As shown in FIG. 4A, the VRNA healthcare training simulator 20 employs the sign-in page GUI 202 to control, e.g., limit, access to the simulator 20 only to authorized users, e.g., operators 10, entering a registered username and password combination at fields, shown generally at 203, including a username field 203A and a password field 203B, respectively, on the sign-in page GUI 202. In one embodiment, registered username and password combinations are maintained within the data store 150. It should be appreciated that while a username and password combination is required to gain access to the simulator 20, it is within the scope of the present invention to employ other login credentials. Once verified as an authorized user/operator of the VRNA healthcare training simulator 20 by the processing system 50, for example, after a lookup operation is successfully performed by the computing device 52 accessing the data store 150 and locating the entered username-password combination therein, the start-up page GUI 204 is presented. In one embodiment, if user/operator verification is unsuccessful, the sign-in page GUI 202 is re-presented to the operator 10 with an error message exhibited thereon requesting re-entry of the username-password combination. As shown in FIG. 4B, the user/operator 10 may select one of a plurality of navigation elements, shown generally at 206, to select a patient 102, for example a Female Patient element 206A or a Male Patient element 206B, for which the operator 10 intends to provide, or assist in providing, care within the 3-D virtual healthcare training environment 100. As should be appreciated, selections and actions taken by the operator 10 (e.g., by manipulating the avatar 120 and/or virtual hands 122) in providing and/or assisting in providing healthcare in the 3-D virtual healthcare training environment 100 are captured and recorded by the data processing system 50 such that the operator's choice or selection and performance may be monitored, evaluated, graded, and/or scored, as compared to preferred or proper techniques for performing and/or assisting in performing the healthcare task (e.g., in accordance with the performance criteria 156).

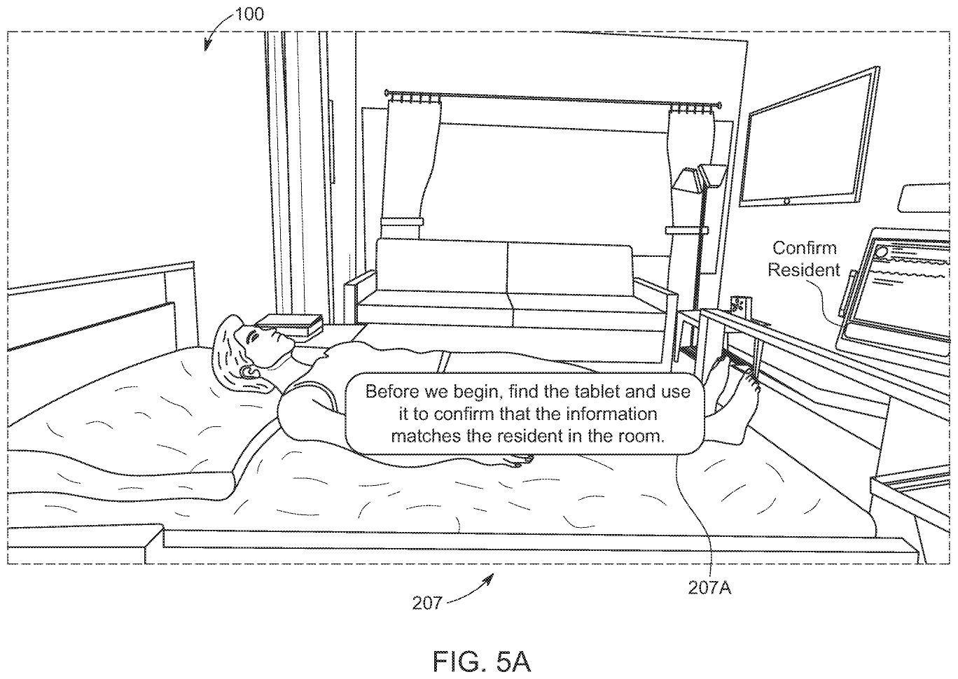

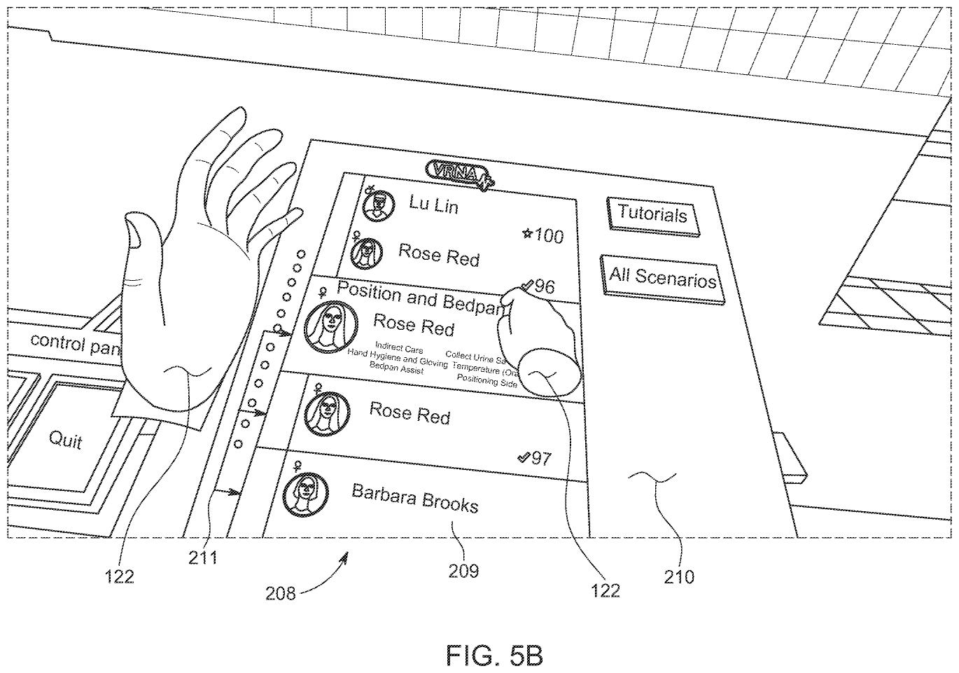

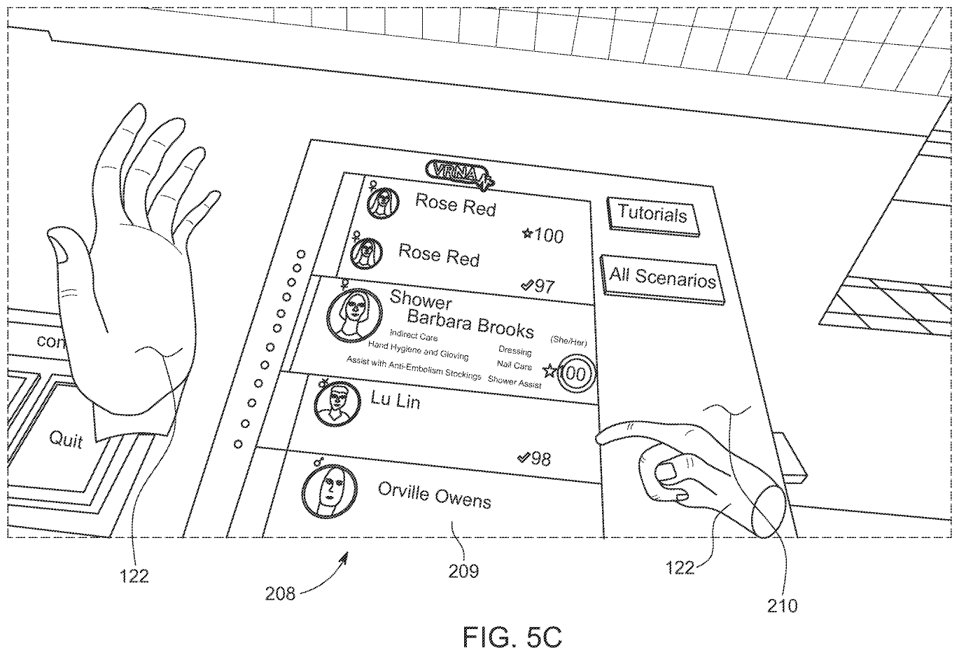

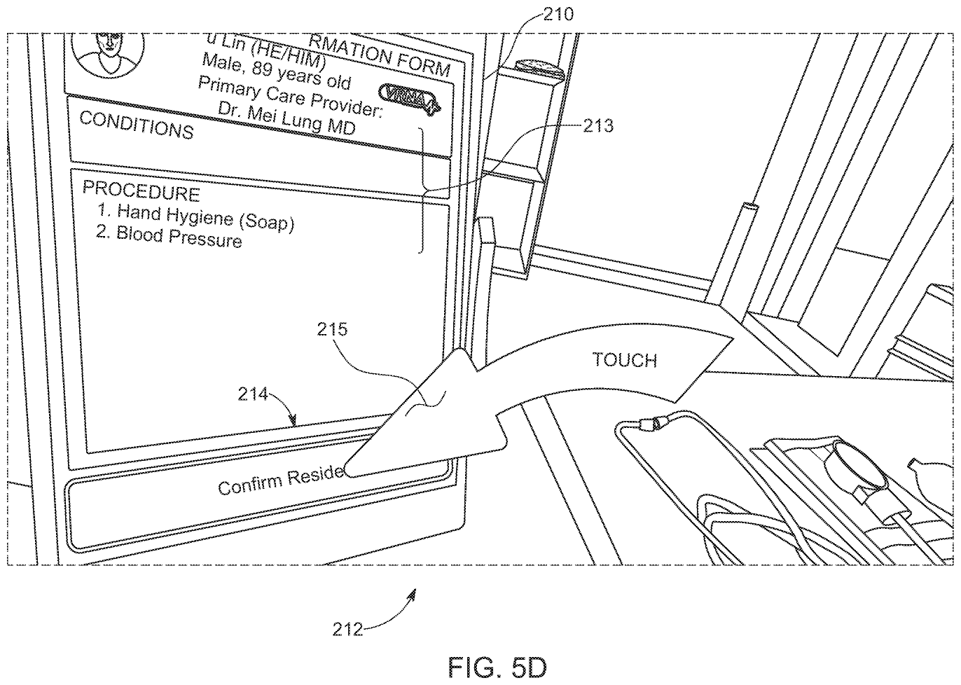

For example, when the operator 10 selects the Male Patient element 206A of the start-up page GUI 204 of FIG. 4B, a series of the GUIs 200 are presented as the operator 10 provides, or assists in providing, healthcare within the training environment 100. In one embodiment, the series of GUIs 200 include, for example, resident/patient information GUIs 207, 208, and 212 of FIGS. 5A to 5D where the operator 10 manipulates the avatar 120 and/or virtual hands 122 with the one or more controllers 60 to review resident/patient information stored within the processing system 50 to ensure the healthcare being provided is to a designated/scheduled one of the residents/patients to receive healthcare. In one embodiment, illustrated in FIG. 5A, the VRNA simulator 20 provides an in-scene instruction 207A tasking the operator 10 to confirm or verify that the resident/patient 102 presented before the operator 10 is the correct resident/patient to receive the scheduled healthcare. In one embodiment, illustrated in FIGS. 5B to 5D, the resident/patient information GUIs 208 and 212 include resident/patient identification information, shown generally at 211, exhibited on a virtual tablet or other portable computing device 210. The resident/patient identification information 211 may include, e.g., a visual depiction, e.g., photograph of each resident/patient, age, a brief description or other identifying characteristics of the resident/patient, a location (e.g., room number), and/or a description of the type or nature of care to be provided. In one embodiment, the resident/patient information 211 is exhibited on the virtual computing device 210 as, for example, a scrollable list of entries 209 as depicted on the GUI 208 (FIGS. 5B and 5C). As shown in FIGS. 5A and 5B, the operator 10 may manipulate the avatar 120 and/or virtual hands 122 to scroll or page through the resident/patient information 211 within the exhibited list 209 on the tablet 210. Once the operator 10 locates the appropriate resident/patient to receive the scheduled healthcare within the exhibited list 209, the operator 10 selects the resident/patient entry, e.g., with a finger tap as shown in FIG. 5C. Once selected, the processing system 50 responds by exhibiting more detailed resident/patient information, shown generally at 213 on GUI 212 of FIG. 5D. In one embodiment, the more detailed information includes, e.g., additional identifying information on the patient/resident, his/her conditions, assigned healthcare practitioners (e.g., primary care provider), and/or healthcare tasks to be performed. In one embodiment, once the operator 10 verifies that the resident/patient is the proper one to receive care, the operator 10 performs a “confirmation” operation 214 as shown in FIG. 5D. In one embodiment, the VRNA healthcare training simulator 20 includes in-scene visual aids or banners 201 and 215 that instruct the operator 10 (“Touch Me” or “Touch” instruction) to complete an operation such as, for example, the sign-in operation as shown on GUI 202 of FIG. 4A and/or the confirmation operation as shown on GUI 212 of FIG. 5D.

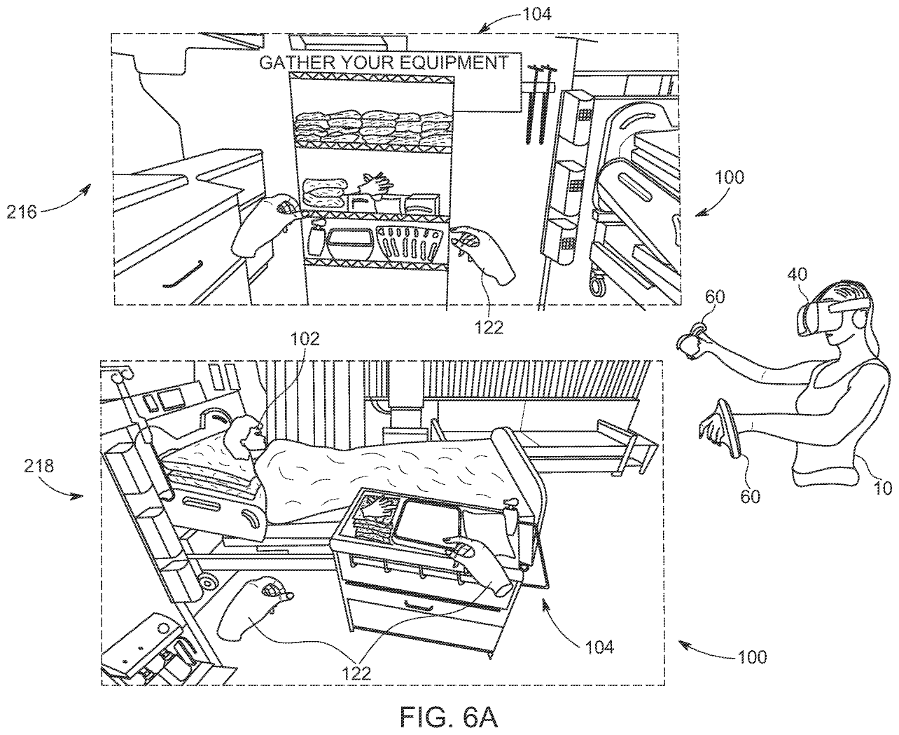

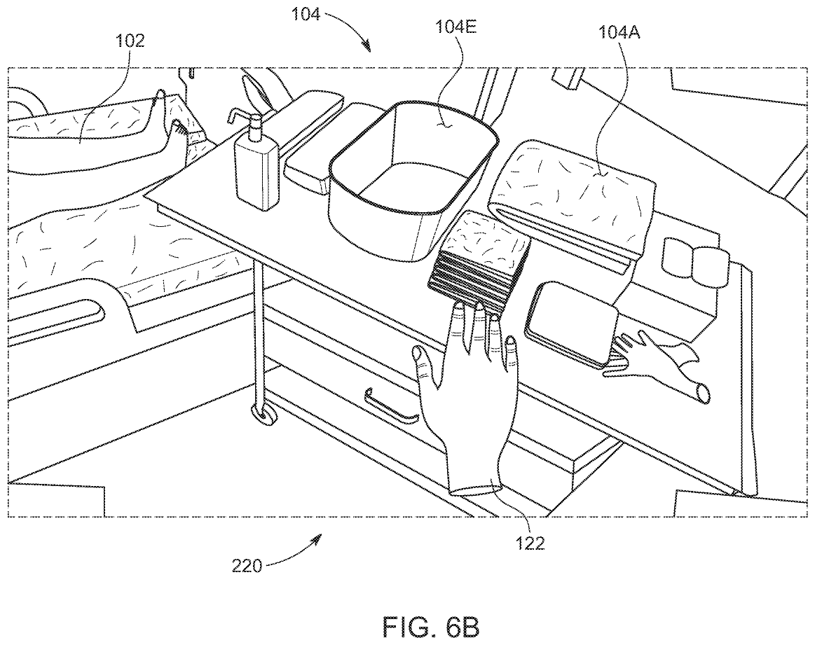

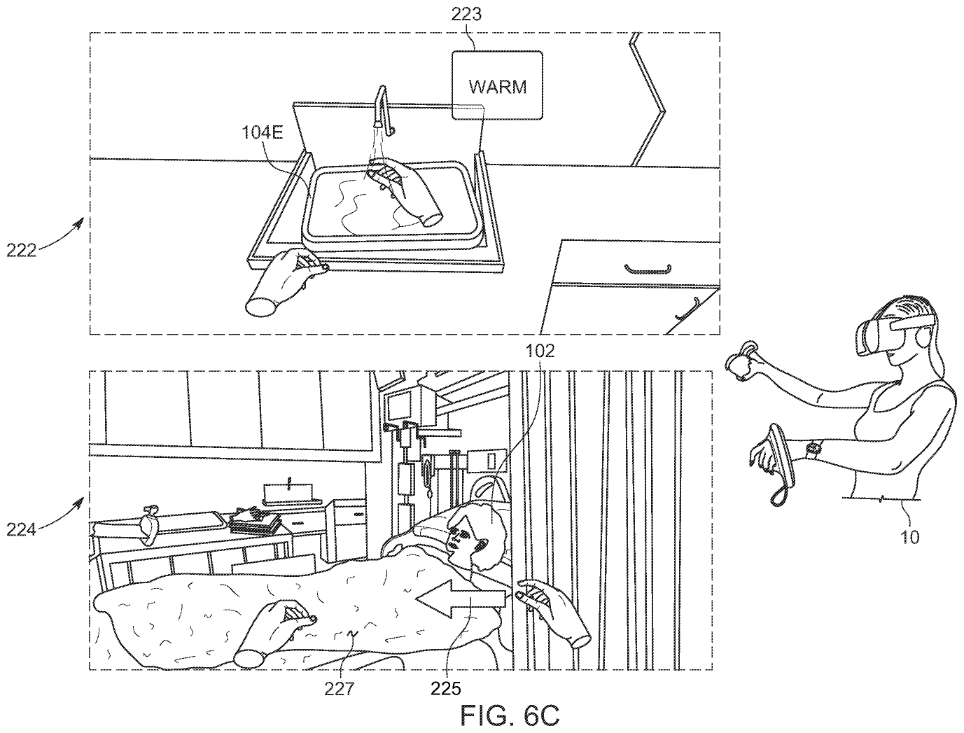

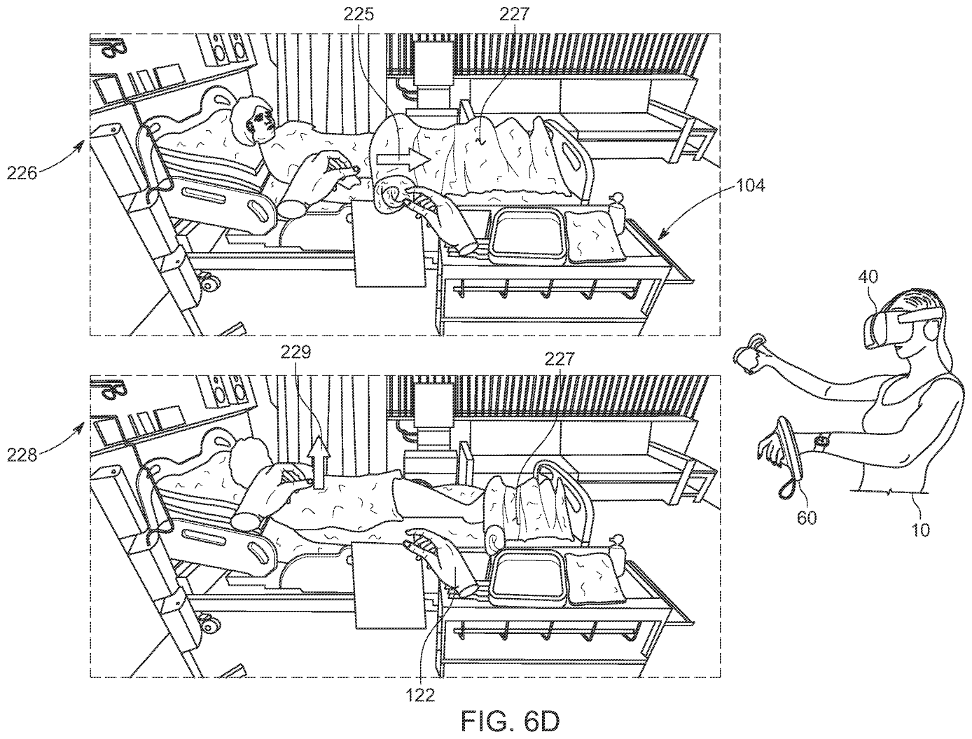

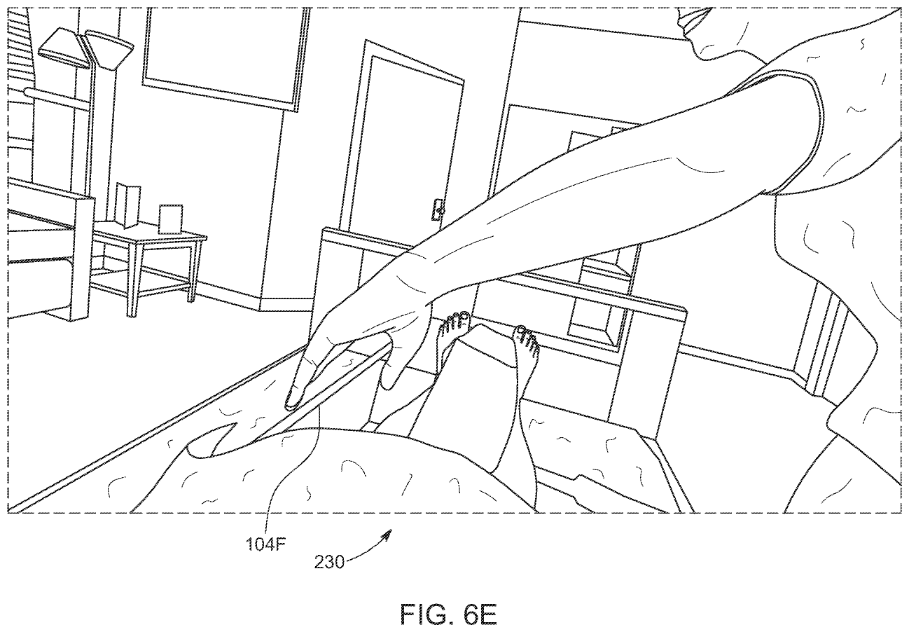

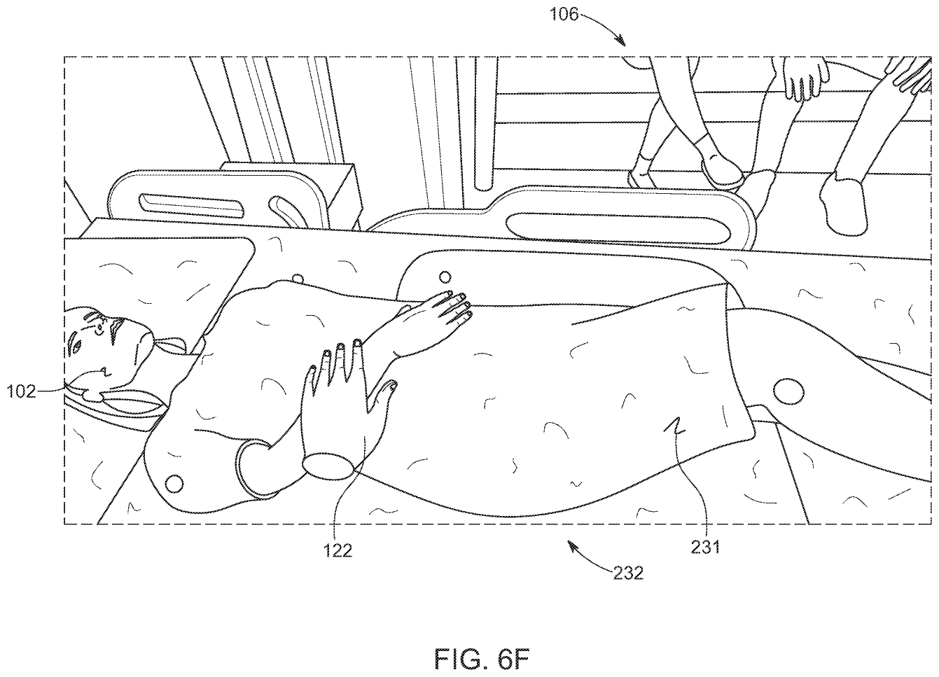

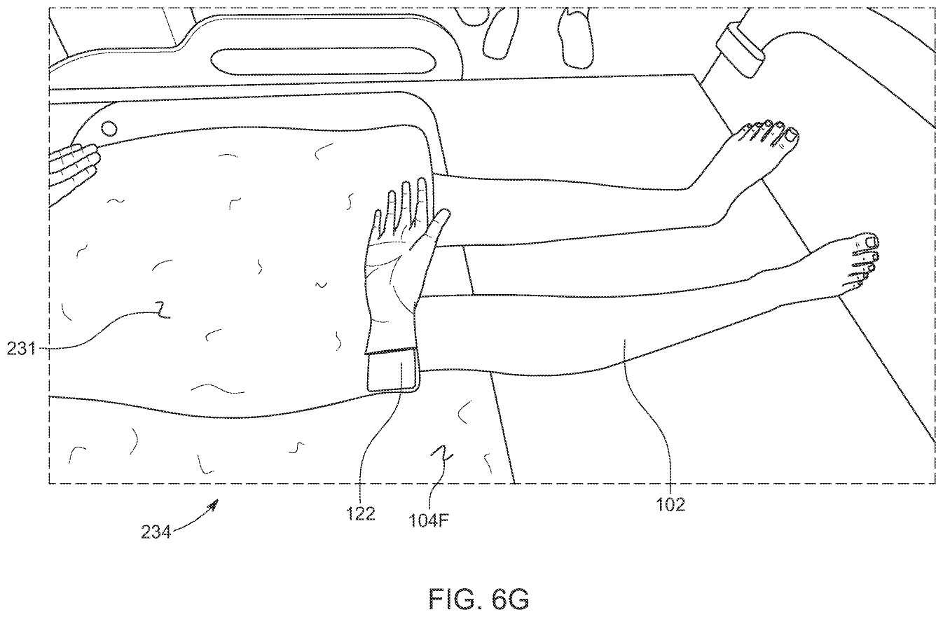

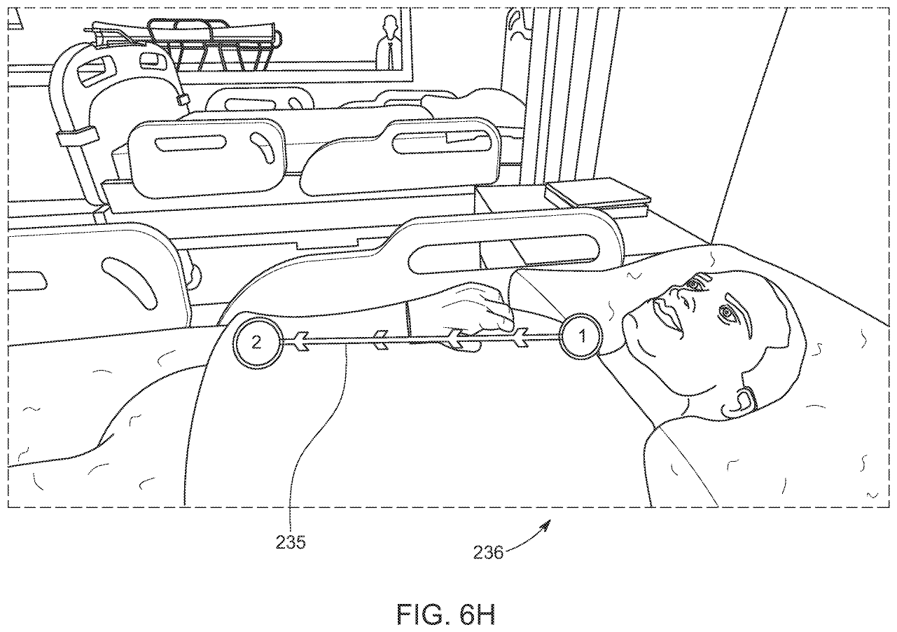

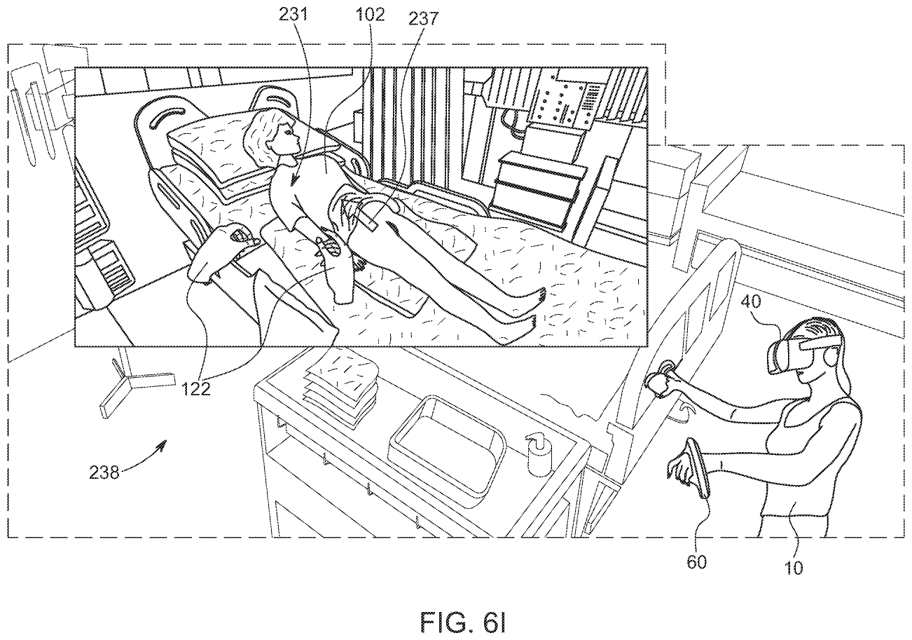

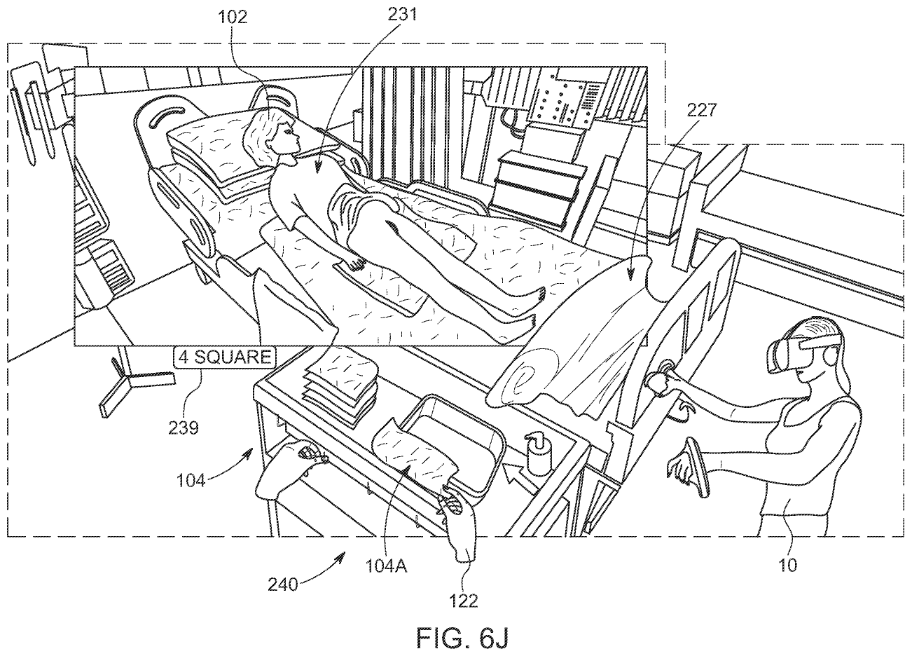

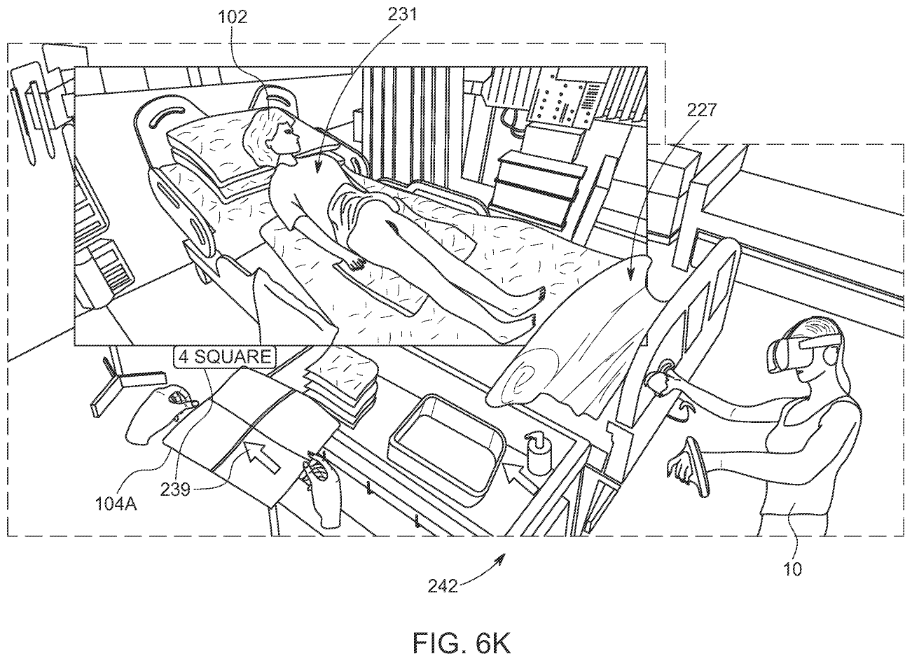

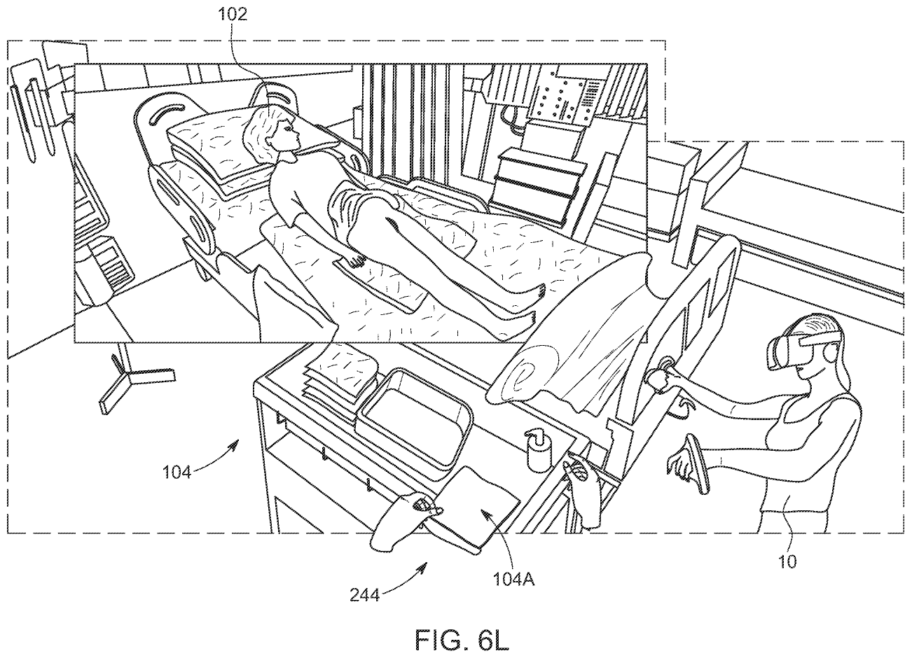

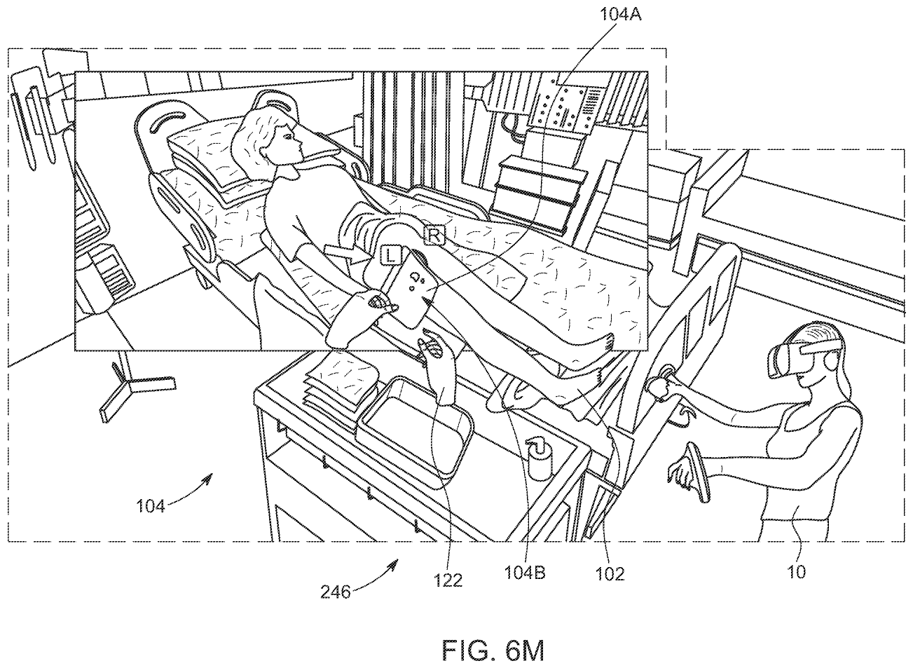

In one embodiment, the series of GUIs 200 further include, for example, GUIs 216 and 218 of FIG. 6A where the operator 10 manipulates the avatar 120 and/or virtual hands 122 with the one or more controllers 60 to gather tools, equipment, and supplies 104 as he/she prepares to provide healthcare to the patient 102. In one embodiment, the processing system 50 of the VRNA healthcare training simulator 20 exhibits an in-scene visual aid, e.g., banner 217, that instructs the operator 10 to complete an operation to “Gather Your Equipment” for performing a healthcare task.

As shown in FIGS. 6A and 6B within GUIs 216, 218, and 220, tools, equipment, and supplies 104 are collected from a storage area, shown generally at 219, and brought to a location proximate the patient 102. In this instance, the health care procedure to be completed by the operator 10 includes cleaning/bathing the patient 102. As shown in GUIs 222, 224, 226, and 228 of FIGS. 6C and 6D, the cleaning/bathing procedure includes gathering clean, warm water in a container 104E. As shown, in one embodiment, the training simulator 20 provides in-scene visual aids 223 and 225, e.g., a sensory indication, instruction, and/or guidance, that the operator 10 should ensure that the water gather is of a preferred “warm” temperature 223 and perform a task (e.g., remove a blanket 227 in a direction indicated by arrow 225) to expose the patient 102 for treatment and care. It should be appreciated that the present invention is not limited in this regard and that it is within the scope of the present invention to employ other sensory displays, icons, and the like, to highlight and/or reinforce instruction, guidance, and/or deficiencies in performance to the operator 10 (e.g., healthcare trainee). As shown in FIGS. 6C and 6D, the operator 10 then proceeds to the patient 102 to prepare him/her to be bathed.

As shown in GUIs 224, 226, 228, 230, 232, 234, 236, and 238 of FIGS. 6C, 6D, 6E, 6F, 6G, 6H, and 6I, respectively, the healthcare procedure includes removing any blankets 227 and/or clothing 231 covering the patient 102 to provide access to areas to be cleaned. As also shown in GUI 228 of FIG. 6D, as necessary, the operator 10 may need to reposition the patient 102 to access the areas to be cleaned, and as illustrated in GUI 228, in one embodiment the simulator 20 may provide an in-scene visual aid 229 (e.g., a sensory indication that the operator 10 should roll the patient in a certain direction as indicated by an arrow 229). As shown in GUI 230 of FIG. 6E, the operator 10 manipulates the avatar 120 and/or its virtual hands 122 with the one or more controllers 60 to grab a sheet, pad, or liner 104F beneath the patient 102 to assist with the rolling of the patient 102 to access an area needing cleaning or care.

As also shown in GUI 232, 234, 236, 238, 240, and 242 of FIGS. 6F, 6G, 6H, 6I, 6J, 6K, and 6L, the operator 10 may need to remove or reposition clothing 231 to access the areas to be cleaned and, in one embodiment, the simulator 20 may provide an in-scene visual aid 235 or 237 (e.g., a sensory indication) that the operator 10 should move the patient's clothing 231 in a certain direction as indicated by a series of arrows 235 from a starting point, indicated by an encircled 1, to an end point, indicated by encircled 2, or by a single arrow 237 (FIGS. 6H and 6I). As should be appreciated, the operator's technique in removing blankets 227, moving clothing 231, or other obstructions and/or repositioning the patient 102 to provide access to the areas to be cleaned, is monitored and evaluated by the training simulator 20, for example, in terms of effectiveness as well as minimizing discomfort to the patient 102 being treated. One evaluation metric includes providing comfort to a resident/patient 102 accordingly, as shown on GUI 232 of FIG. 6F, one positive action is the operator 10 informing the resident/patient 102 of the action that the operator 10 is about to take prior to beginning the action. As shown in GUIs 232 and 234 of FIGS. 6F and 6G, the cleaning procedure may include perineal care on private areas of the patient and may be carried out as one or more visitors 106 or other third parties are present. In view thereof, the operator's performance of treatment and care while providing privacy for the patient 102 is also an evaluation metric.

As shown in GUIs 240, 242, 244, and 246 of FIGS. 6J, 6K, 6L, and 6M, respectively, once access is provided and the patient 102 is in a stable position, the operator 10 manipulates the avatar 120 and/or virtual hands 122 with the one or more controllers 60 to prepare the tools, equipment, and supplies 104 to clean the area of the patient 102. As shown in GUIs 240 and 242 of FIGS. 6J and 6K, the training simulator 20 monitors, evaluates, and as needed reinforces, proper techniques for preparing tools, equipment, and supplies 104 used in providing medical care. For example, in cleaning procedures, a cloth or towel 104A is folded and used in a particular way (e.g., a so-called “4 Square” method as indicated by an in-scene visual aid 239 of GUIs 240 and 242 of FIGS. 6J and 6K) so that a clean portion of the cloth or towel 104A is only used once on a patient and after use, when at least the towel 104A is partially contaminated, the cloth or towel 104A is repositioned so that the contaminated portion does not contact the patient 102 again. As shown in GUI 246 of FIG. 6M, the operator 10 using proper technique ensures that a contaminated portion, shown generally at 104B, of the cloth or towel 104A is not in contact with the patient 102 or other healthcare practitioners including themselves. As should be appreciated, the operator's technique in performing the cleaning procedure is monitored and evaluated by the training simulator 20.

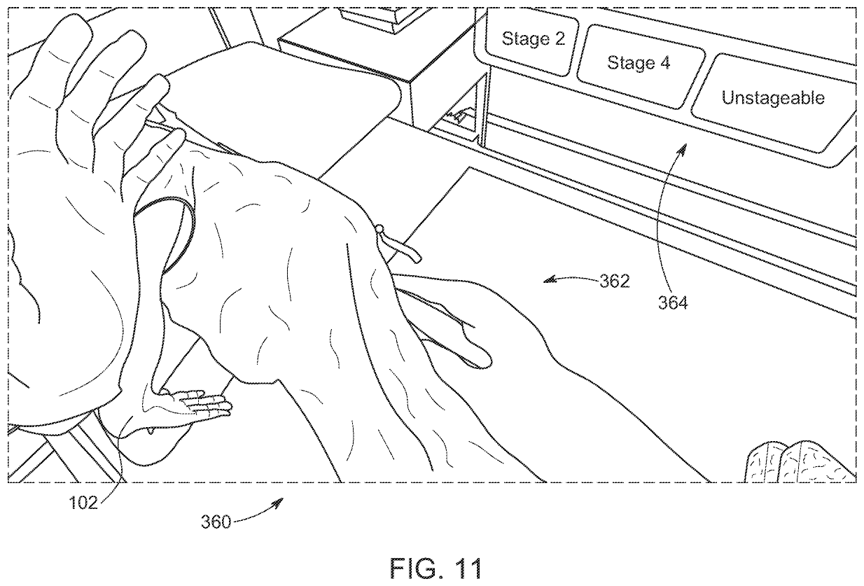

It should be appreciated that one aspect of providing the aforementioned cleaning tasks is for a healthcare practitioner to assess a preexisting or newly developed condition of the resident/patient undergoing care. For example, it is not uncommon for bedridden residents/patients to develop skin ulcers from lack of movement or mobility leading to poor blood flow in areas of his/her body and as a result loss of outer layers of their skin, redness, and in extreme cases, open sores, wounds, and ulcers. In one embodiment, as shown on GUI 360 of FIG. 11, when the operator 10 detects a newly developed or a preexisting condition, shown generally at 362, such as a sore, wound, or skin ulcer, the operator 10 evaluates and records the observed condition. For certain conditions, it is well known to assign a stage or level of severity of the condition. In one embodiment, illustrated on the GUI 360 of FIG. 11, the operator 10 assigns, as shown generally at 364, a stage or notes it is not possible to assign a stage (“unstageable” notation) to the condition 362 within the resident's or patient's medical record. As should be appreciated, the operator's evaluation and recordation of the patient's condition, and any changes thereto, is evaluated as a performance metric by the training simulator 20.

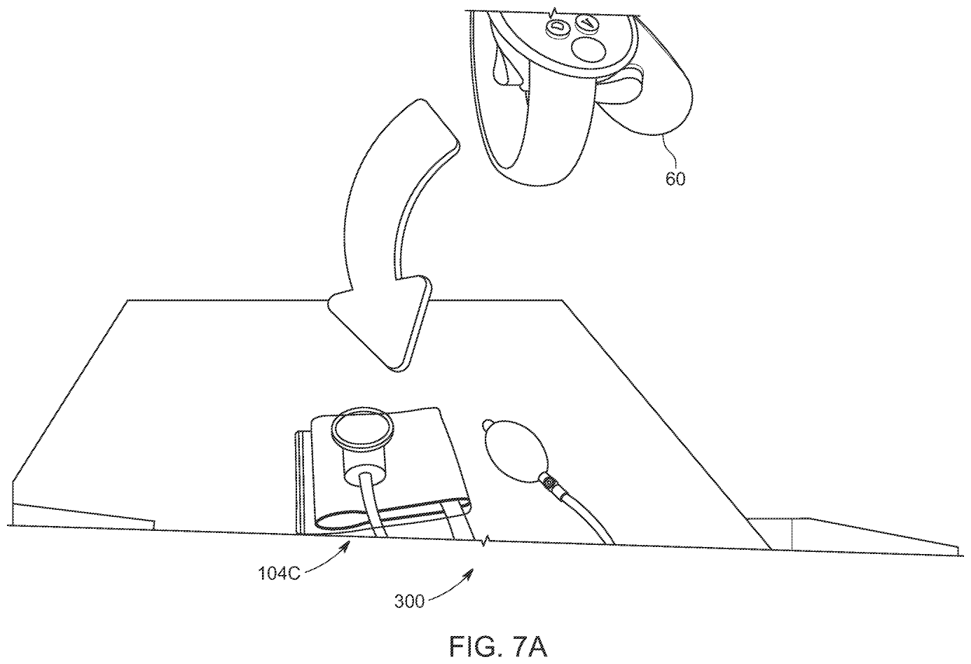

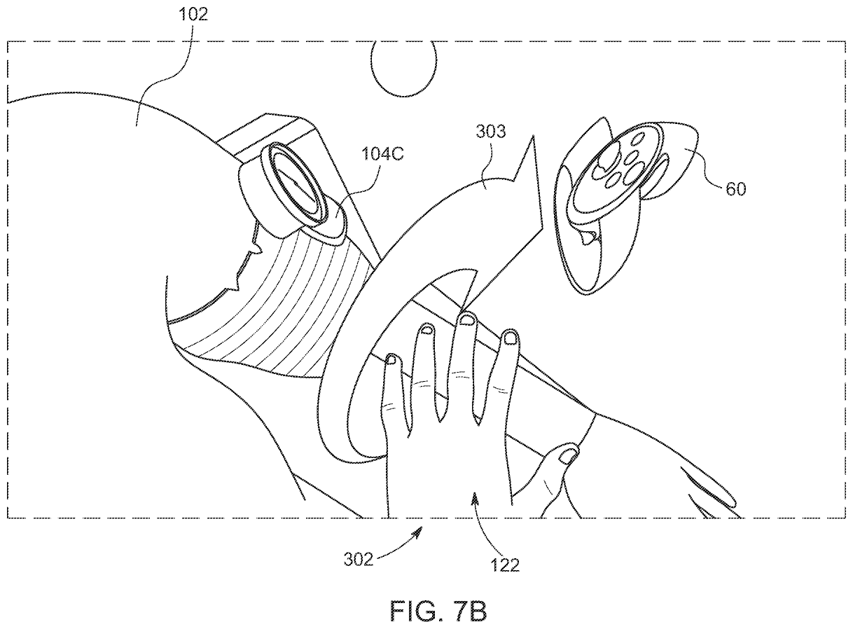

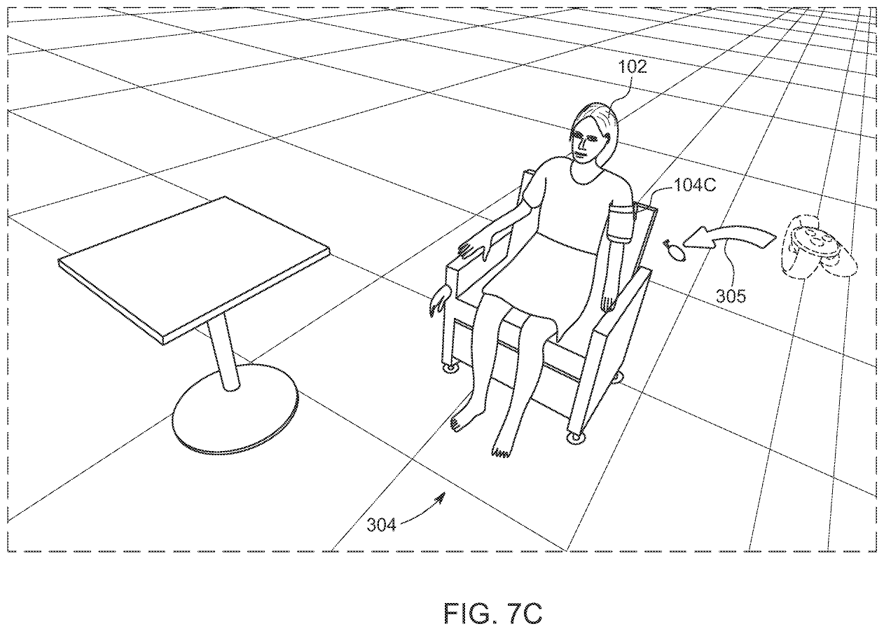

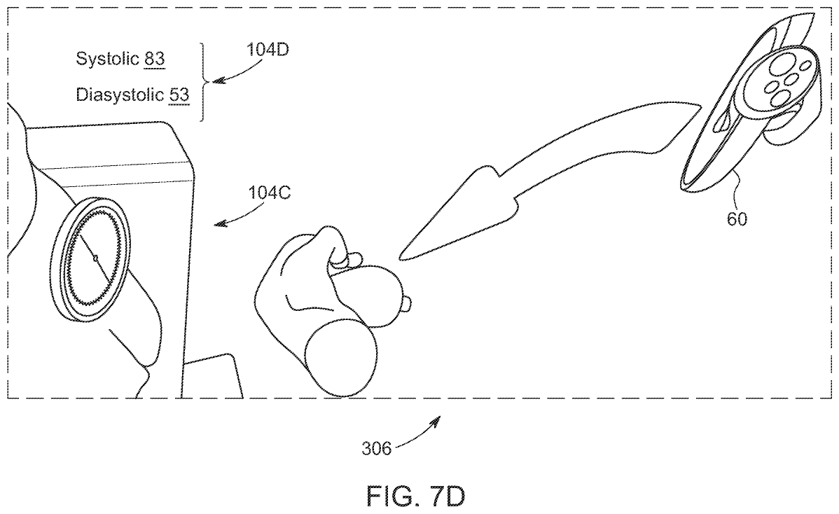

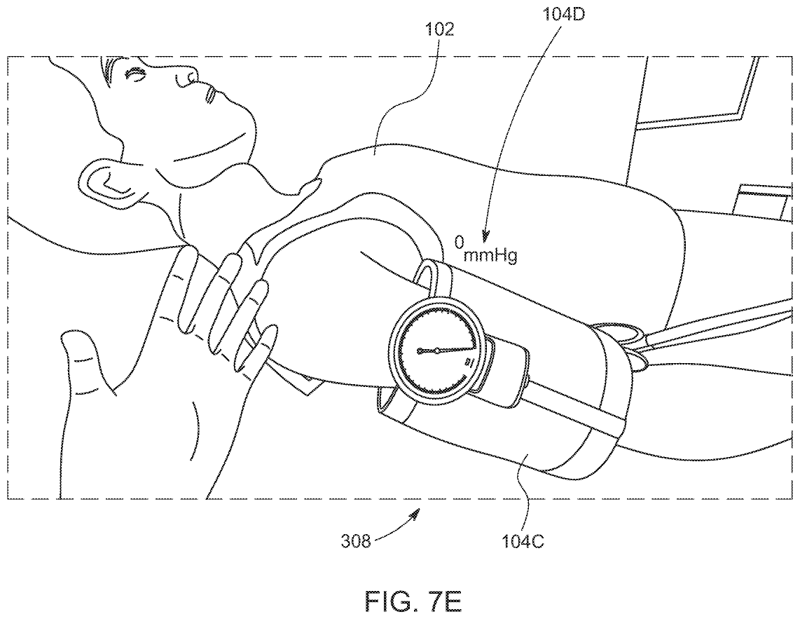

In one embodiment, the VRNA training simulator 20 provides a series of the GUIs 200 to, for example, monitor and evaluate a healthcare trainee (e.g., the operator 10) performing such medical or patient care task as taking, or assisting other medical practitioners taking, a patient's vital signs (e.g., temperature, blood pressure, blood glucose level, blood flow, and the like) and/or being administered medicine. For example, as shown in GUIs 300, 302, 304, 306, and 308 of FIGS. 7A, 7B, 7C, 7D, and 7E, respectively, the training simulator 20 monitors and evaluates a healthcare trainee (e.g., the operator 10) taking the patient's 102 arterial blood pressure. As shown in FIG. 7A, in one embodiment, the training simulator 20 provides a virtual representation of an aneroid sphygmomanometer 104C, which includes an aneroid pressure gauge connected to an inflatable cuff, as one of the objects 104 (e.g., the healthcare tools, equipment, and supplies) used by operators 10 providing care within the 3-D virtual VRNA healthcare training environment 100. As shown in GUI 302 of FIG. 7B, in one embodiment, the training simulator 20 provides an in-scene visual aid 303 (e.g., a sensory indication, instruction, and/or guidance) that the operator 10 should affixed the cuff portion of the aneroid sphygmomanometer 104C about an arm 102A of the patient 102 in a preferred manner. As shown in GUI 304 of FIG. 7C, in one embodiment, the training simulator 20 provides an in-scene visual aid 305 (e.g., a sensory indication, instruction, and/or guidance) as to how the operator 10 should operate the aneroid sphygmomanometer 104C by squeezing a simulated pump portion of the aneroid sphygmomanometer 104C to inflate the cuff portion thereof. As shown in FIGS. 7D and 7E, the aneroid sphygmomanometer 104C measures and outputs, as shown generally at 104D, a patient's arterial blood pressure in readings of, e.g., Systolic and Diastolic values. The operator 10 records in a medical chart for the patient 102, for example, using the tablet 201 (FIG. 5A), not shown. As should be appreciated, the operator's technique in measuring, reading, and recording the output values 104D is monitored and evaluated by the VRNA training simulator 20.

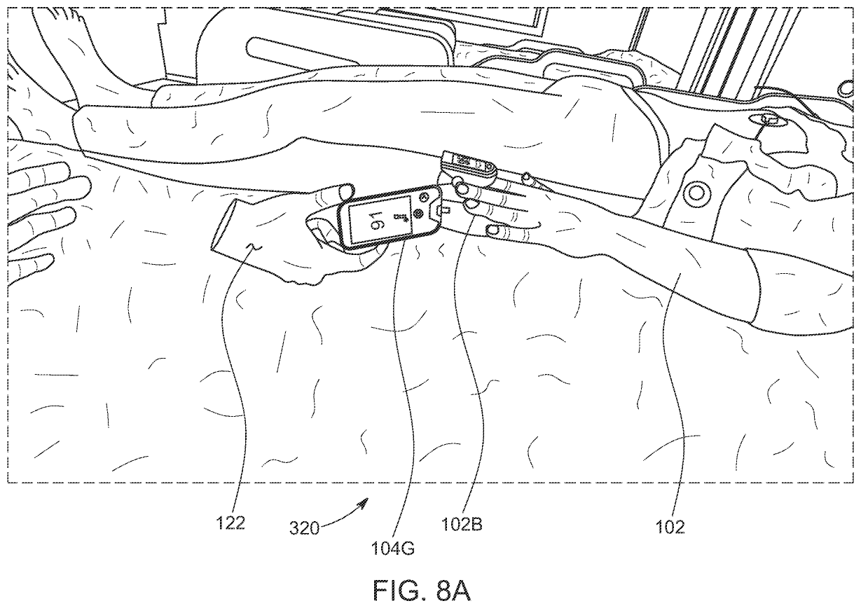

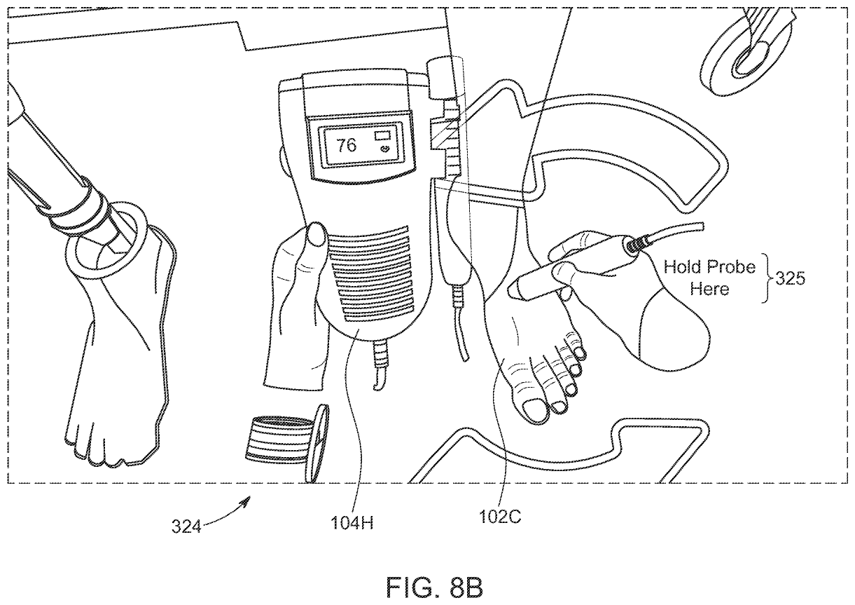

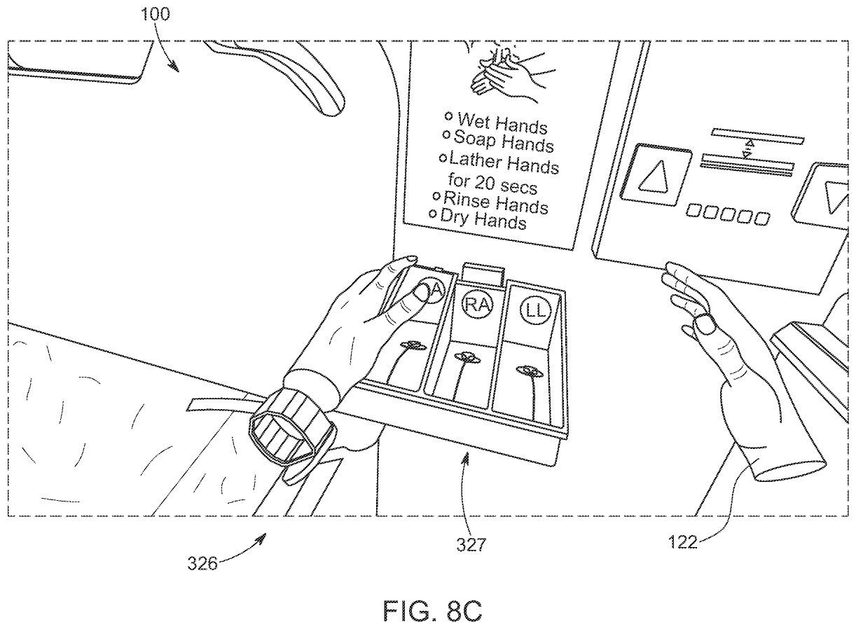

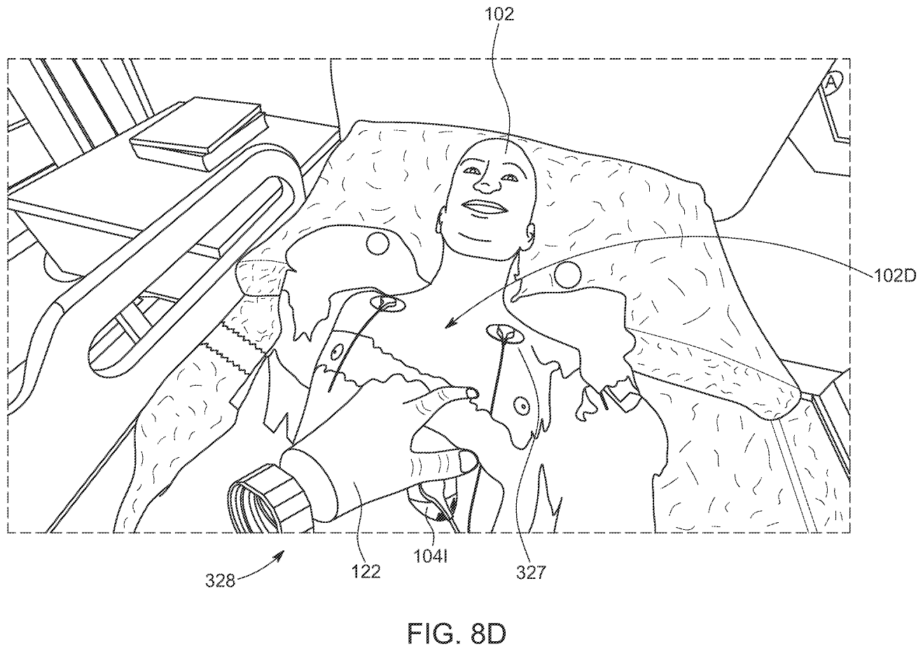

In one embodiment, the VRNA training simulator 20 also provides a series of the GUIs 200 to, for example, monitor and evaluate the healthcare trainee (e.g., the operator 10) measuring, reading, and recording other vital signs of the resident/patient 102 such as, e.g., a patient's blood glucose level at a patient's finger 102B with a simulated glucose meter 104G on a GUI 320 of FIG. 8A or blood flow through a patient's blood vessels with a simulated Dopler ultrasound flow meter 104H on a GUI 324 of FIG. 8B. As shown in FIG. 8B, the processing system 50 of the VRNA healthcare training simulator 20 exhibits an in-scene visual aid, e.g., instruction 325, advising the operator 10 where to locate a probe portion of the flow meter 104H when measuring the blood flow in the resident/patient' s 102 foot 102C. As should be appreciated by those skilled in the art, at times, a degree of set up or instrument configuration is needed to accurately measure a patient's vital signs. For example, when measuring electrical activity of a patent's heart with, e.g., an ECG (electrocardiogram) also referred to as an EKG (elektrokardiogramm; German language spelling) device, it may be necessary to affix sensors or leads about the patient's chest. Location of the sensors or leads can be important for accurate measurement. As shown on GUIs 326 and 328 of FIGS. 8C and 8D, leads, shown generally at 327, of an ECG device 104I are affixed to a chest 102D of a patient 102 so that the electrical activity of the patent's heart can be measured and recorded. It should be appreciated that while a subset of exemplary healthcare tasks have been defined above, it is within the scope of the present invention to implement any number of healthcare tasks including the use of numerous the healthcare tools, equipment, and supplies to sense and measure conditions of a patient and/or provide needed or desired health care. For example, while the use of the Dopler ultrasound flow meter 104H is described, it is within the scope of the present invention to simulate and train an operator 10 on the use of other medical imaging equipment such as, for example, devices including ultrasound, echocardiography, magnetic fields (MRI), electromagnetic radiation (conventional two- and three-dimensional X-ray, tomography, CT scan, PET scans, fluoroscopy), and breast thermography, whether in mobile or fixed form factors. Accordingly, the present invention should not be limited by the illustrated embodiments.

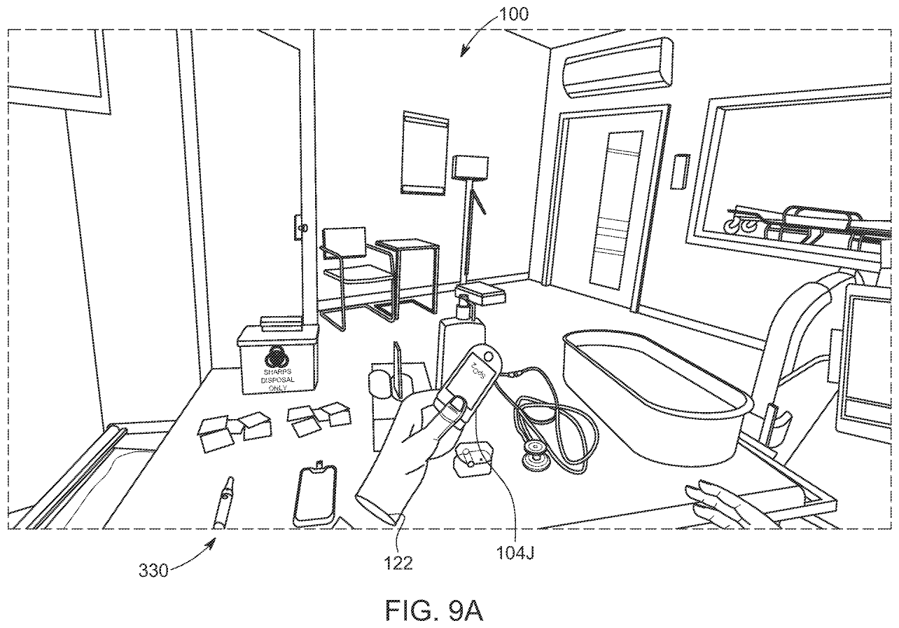

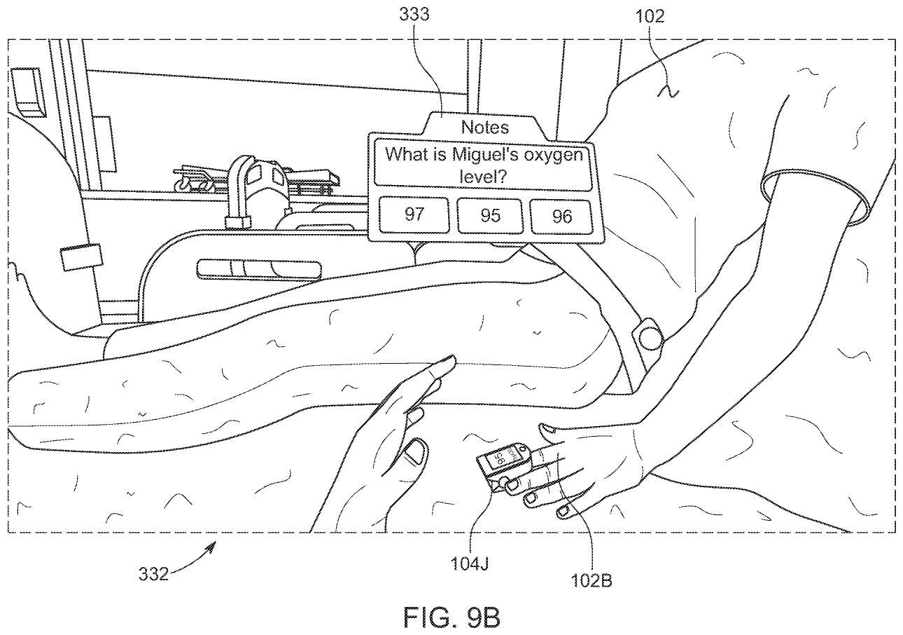

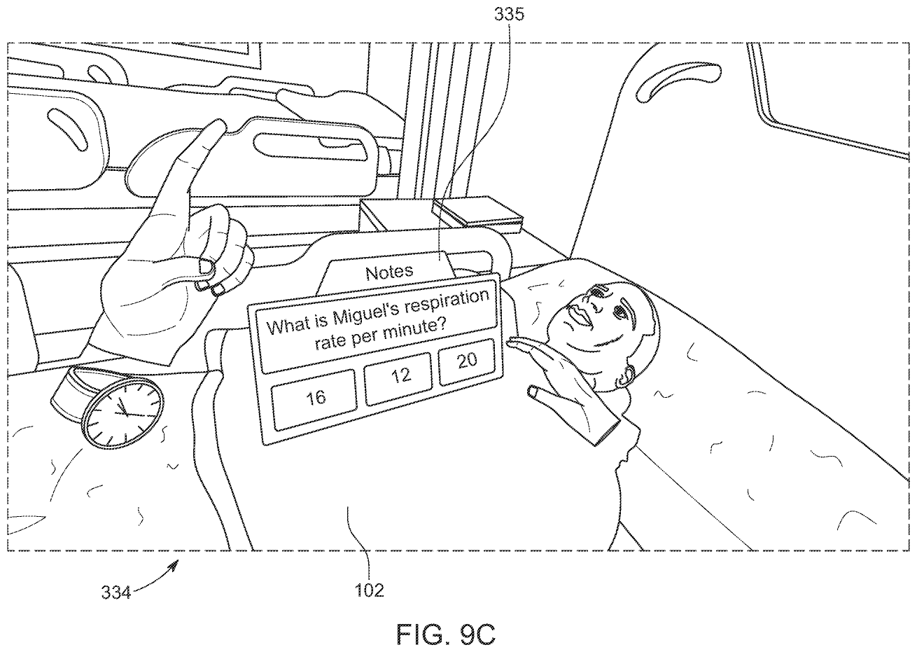

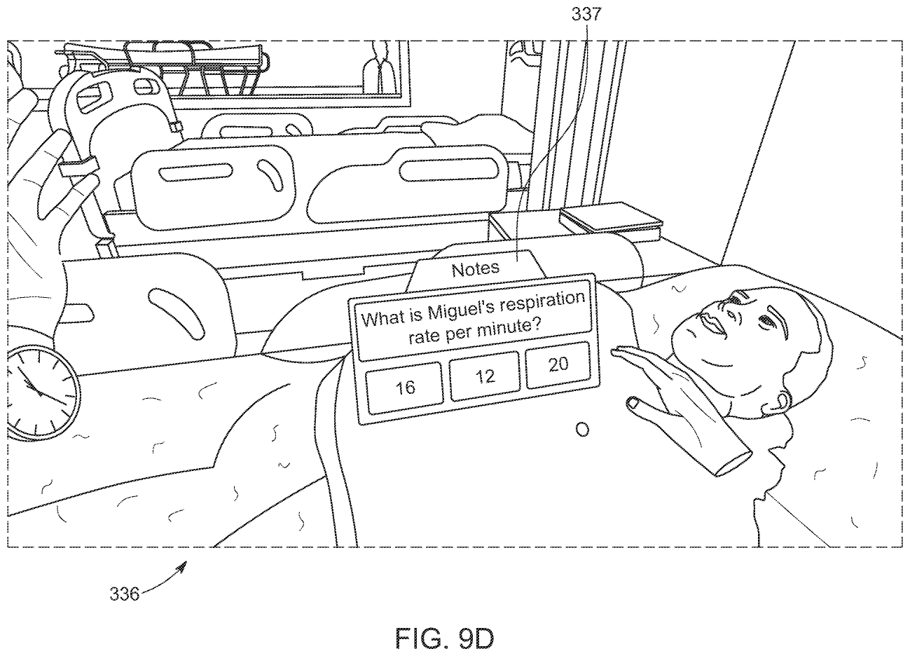

In one embodiment, the VRNA training simulator 20 may introduce one or more tests or quizzes to, for example, periodically evaluate the healthcare trainee (e.g., the operator 10) knowledge and skill in completing a healthcare task. For example, as illustrated in FIGS. 9A to 9E, the processing system 50 of the VRNA healthcare training simulator 20 instructs the trainee/operator 10 to gather a meter and to measure a vital sign of a patient. As shown in a GUI 330 of FIG. 9A, in response to the instruction the operator 10 manipulates the avatar 122 with the controllers 60 to first collect a pulse-oximeter device 104J and then to proceed to a subject resident/patient 102. As shown in GUIs 332, 334, and 336 of FIG. 9B, 9C, and 9D, after the operator 10 affixes the pulse-oximeter device 104J to the patient's finger 102B, the training simulator 20 queries the operator to measure vital signs and record measurements observed. For example, at a Notes block 333 on GUI 332 of FIG. 9B, the simulator 20 asks the operator 10 to respond to a question “What is Miguel's oxygen level?” and offers three (3) possible values, e.g., “97”, “95”, and “96”. The operator's performance is evaluated based on his/her correct or incorrect response to the question presented. Similarly, at a Notes block 335 on GUI 334 of FIG. 9C, the simulator 20 asks the operator 10 to respond to a question “What is Miguel's respiration rate per minute?” and again offers three (3) possible values, e.g., “16”, “12”, and “20”. As shown at a Notes block 337 on GUI 336 of FIG. 9D, in one embodiment, the simulator 20 revises the Notes block 335 to indicate the operator's response, different color “12” option in the Notes block 337 as compared to the Notes block 335.

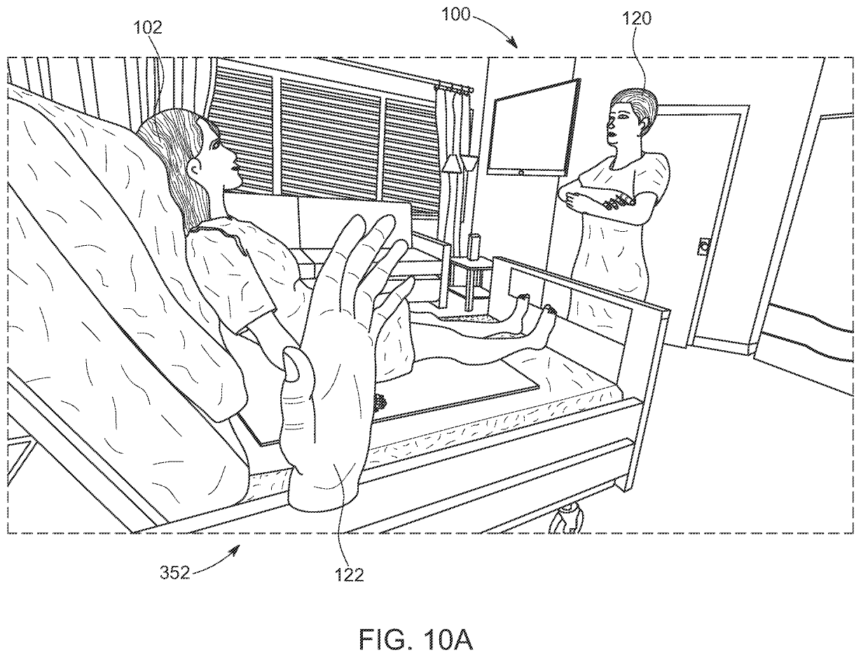

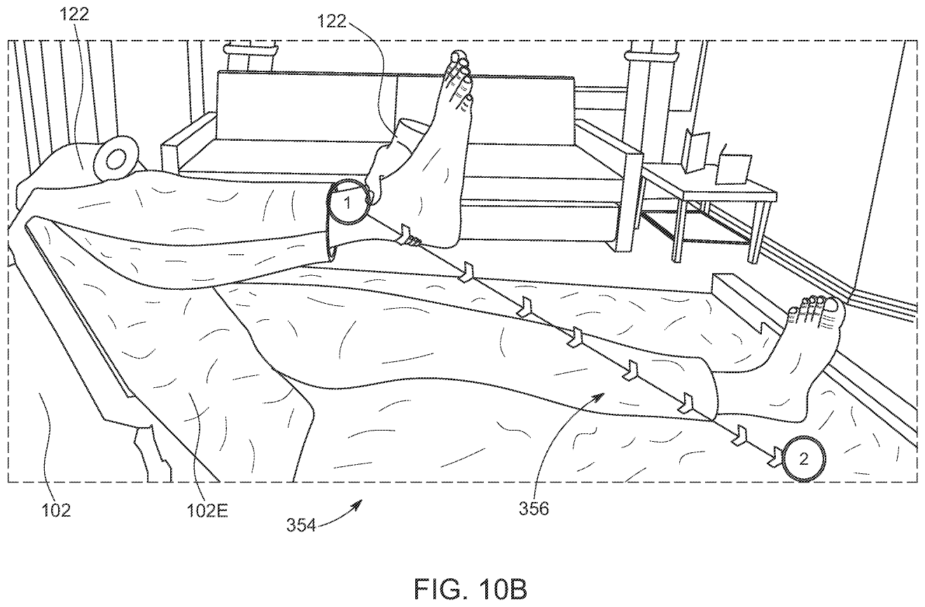

In one embodiment, the healthcare provided to residents/patients may include, for example, physical therapy. For example, as shown on GUIs 350 and 354 of FIGS. 10A and 10B, the operator 10 manipulates the avatar 120 and/or virtual hands 122 with the one or more controllers 60 to supervise a patient that is performing or is assisted in performing, exercises to, for example, increase the patient's heartrate, promote blood flow, assist in improving the patient's mental outlook, or provide other perceived advantages to the patient 102. For example, as depicted in FIG. 10B, the operator 10 manipulates the virtual hands 122 to, in turn, move the patient's leg 102E from a resting position to a flexed or bent position, indicated at point 1, back to a straight position, indicted at point 2, along a path indicated by arrow 356. As is known in healthcare practice, this exercise is done to move muscles in limbs of the patient to help strengthen muscle and to minimize muscle atrophy.

As should be appreciated from the description presented herein, the VRNA healthcare training simulator 20 implements the 3-D virtual healthcare training environment 100 for training and re-training healthcare trainees operating the system to gain and/or further refine a plurality of healthcare skills. For example, the healthcare skills within the VRNA healthcare training simulator 20 include, but are not limited to, the following:
1. Airway Obstruction
2. Assisted Ambulation
3. Assisted Anti-Embolism Stockings
4. Assisted Bedpan
5. Assisted Meal
6. Assisted Shower
7. Assisted Transfer
8. Assisted Urinal
9. Bed Bath
10. Catheter Care
11. Collect Urine Sample
12. Denture Care
13. Dressing
14. Hand Hygiene and Gloving
15. Indirect Care
16. Making an Occupied Bed
17. Making an Unoccupied Bed
18. Massage (Back)
19. Measure Blood Pressure
20. Measure Height and Weight
21. Measure Intake and Output
22. Measure Pulse
23. Measure Respiration
24. Measure Temperature (Axillary)
25. Measure Temperature (Oral)
26. Measure Temperature (Tympanic)
27. Mouth Care (Conscious)
28. Mouth Care (Unconscious)
29. Nail Care
30. Passive Range of Motion
31. Perineal Care
32. Positioning (Side)
33. Positioning (Supine)
34. Shaving
35. Administer AED (Automated External Defibrillator)
36. Administer CPR (Cardiopulmonary Resuscitation)
37. Administer Mechanical CPR (Cardiopulmonary Resuscitation)
38. Airway Suctioning
39. Amputated Limb Care
40. Assisted Complicated Childbirth
41. Assisted Childbirth
42. Assisted Medications
43. Auto Injector
44. Automated Transport Ventilators
45. Bleeding Control
46. BVM Ventilation (Bag-Valve-Mask)
47. Dead Body Care
48. Humidified Oxygen
49. Manual Airway Techniques
50. MAST/PASG (Medical Anti-Shock Trousers, Pneumatic Anti-Shock Garments)
51. Measure Blood Glucose
52. Measure Blood Pressure (Manual)
53. Measure Blood Pressure (Automated)
54. Measure Pulse (Apical)
55. Measure Pulse (Dorsalis)
56. Measure Pulse (Oximeter)
57. Nasal Airways
58. Oxygen Therapy
59. Phlebotomy
60. Place Electrocardiogram (EKG)
61. Positioning (Log Roll)
62. Prosthesis Care
63. Record Patient Medical Information
64. Spinal Immobilization
65. Splinting
66. Therapeutic Massage
67. Tourniquet
68. Ulcer Identification
69. Venturi Mask
70. Wound Care

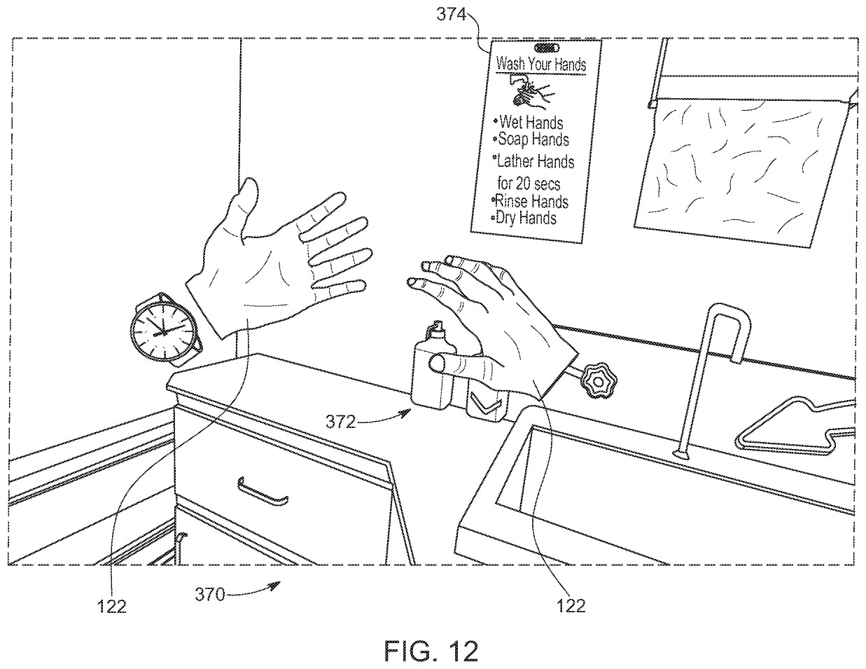

As can be appreciated by those skilled in healthcare practice, hand hygiene is important as it prevents the spread of germs thus protecting both the caregiver as well as those persons receiving care from the caregiver. Accordingly, as shown in GUI 370 of FIG. 12, the VRNA training simulator 20 monitors and records the number of times and effectiveness of the operator 10 undertaking a task of cleaning their own hands before or after providing care. In one embodiment, the simulator 20 may track how much anti-bacterial soap or cleaners, shown generally at 372, the operator 10 applies to his/her virtual hands 122, if at all, as well as a duration of the washing procedure. In one embodiment, the simulator 20 exhibits signage 374 within the virtual environment 100 providing instruction or direction to the operator 10 as to effective washing procedures.

In one embodiment, the VRNA training simulator 20 may capture and record (e.g., via the tracking sensors 44 and 62) one or more paths of travel of the one or more controllers 60 as the operator 10 manipulates one of the objects 104 (e.g., the healthcare tools, equipment, and supplies) used by operators 10 in providing care, and/or of the HMDU 40 as an indication of the operator's physical movement within and about the 3-D virtual healthcare training environment 100. In one embodiment, the training simulator 20 may generate, for example, in a review and/or evaluation mode, a line as a visual indication of the one or more captured and recorded paths of travel of the objects 104 and/or the operator 10 to demonstrate the position and/or orientation thereof as a performance measurement tool. In one embodiment, such a performance measurement tool may be used, for example, to demonstrate proper and efficient, and/or improper and inefficient performance of healthcare procedures conducted by the operator 10. In one embodiment, the visual indication of two or more paths of travel may be color coded or otherwise made visually distinct, may include a legend or the like, depicting and individually identifying each of the paths of travel, and/or may include one or more visual cues (e.g., a starting point, cone, arrow, or icon, numeric or alphanumeric character, and the like) illustrating aspects of the paths of travel such as, for example, speed, direction, orientation, and the like.

As should be appreciated, it is within the scope of the present invention to provide more and/or different sensory indications (e.g., visual graphs and icons, audio and/or tactile indications) to illustrate, for example, both favorable and/or unfavorable aspects of the performance of healthcare procedures by the operator 10 (e.g., healthcare trainee) within the 3-D virtual healthcare training environment 100. The inventors have discovered that this in-process, real-time sensory guidance (e.g., the visual, audio and/or tactile indications) can improve training of the operator 10 by influencing and/or encouraging in-process changes by the operator 10 such as positioning (e.g., proximity and/or angle) of the one or more controllers 60 in relation to the patient 102. As can be appreciated, repeated performance at, or within a predetermined range of, optimal performance characteristics develops and/or reinforces skills necessary for performing a skill-oriented task. Accordingly, the training simulator 20 and its real-time evaluation and sensory guidance toward optimal performance characteristics are seen as advantages over conventional training techniques. Furthermore, in some embodiments, the performance characteristics associated with the operator 10 and/or the quality characteristics associated with the healthcare virtually rendered thereby may be used to provide a measure or score of a capability of the operator 10, where a numeric score is provided based on how close to optimum (e.g., preferred, guideline, or ideal) the operator 10 is for a particular tracked procedures and the like.

As described above, the healthcare training simulator 20 tracks, captures or records, and utilizes various cues and sensory indications to exhibit both favorable and/or unfavorable aspects of the healthcare procedures being performed by the operator 10. In one aspect of the invention, the simulator 20 evaluates an operator's performance and the tools, equipment, and supplies 104 used, as well as the controller 60 movement (e.g., speed, direction or path, orientation, distance), to a set of performance criteria established by, for example, the instructor or certification agent 12 and/or healthcare industry standards of acceptability. In one embodiment, the training simulator 20 based evaluation yields scores and/or rewards (e.g., certification levels, achievement badges, and the like) highlighting the operator's progress and/or results as compared to the set of performance criteria and, in one embodiment, as compared to other healthcare trainees. The scoring may be determined and/or presented both on an in-process and/or on a completed task basis. As noted above, the scoring may include evaluations of operator's actions in manipulating the patient 102 and/or objects 104 by movement of the one or more controllers 60 (e.g., speed, orientation, distance) as the operator 10 performs a healthcare procedure and tasks therein as well as the operator's performance with respect to other parameters such as, for example, elapsed time, efficiency, resulting patient condition and/or improved condition (e.g., perceived good and bad results).

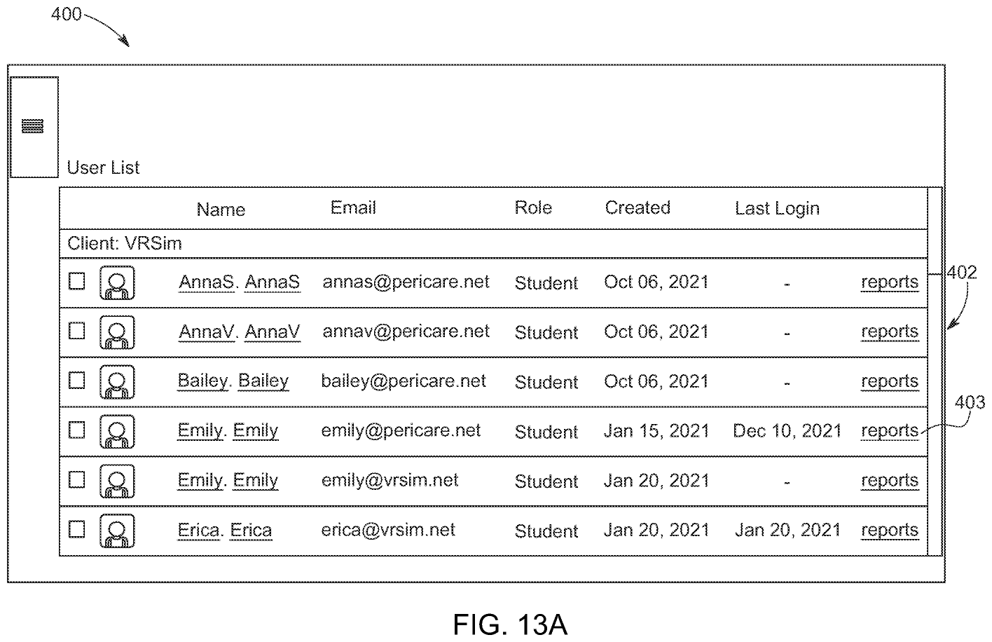

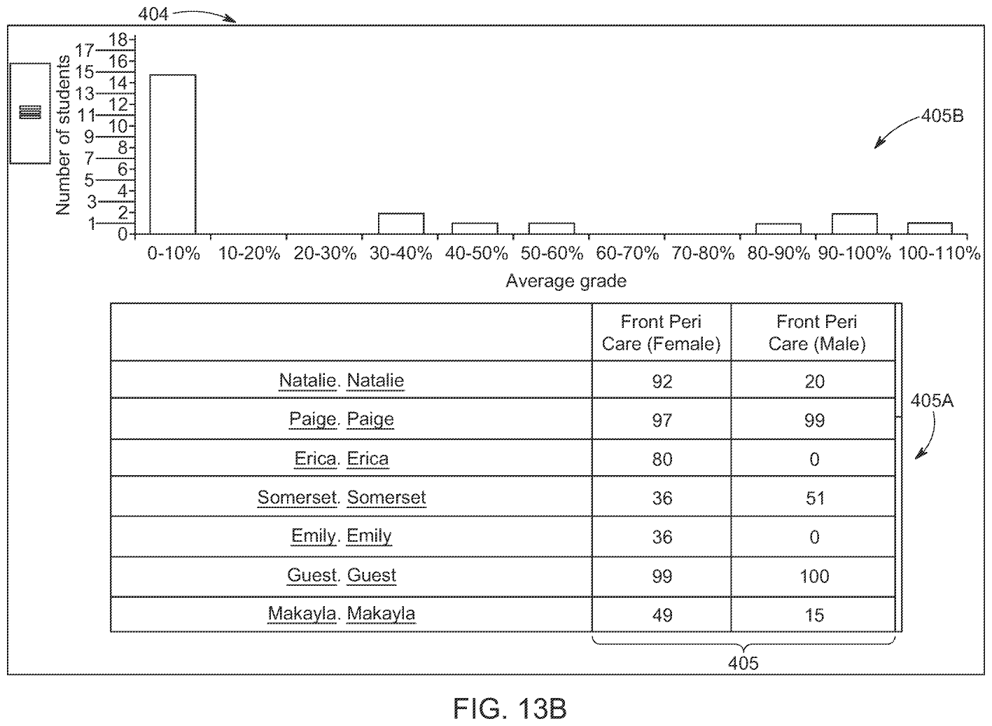

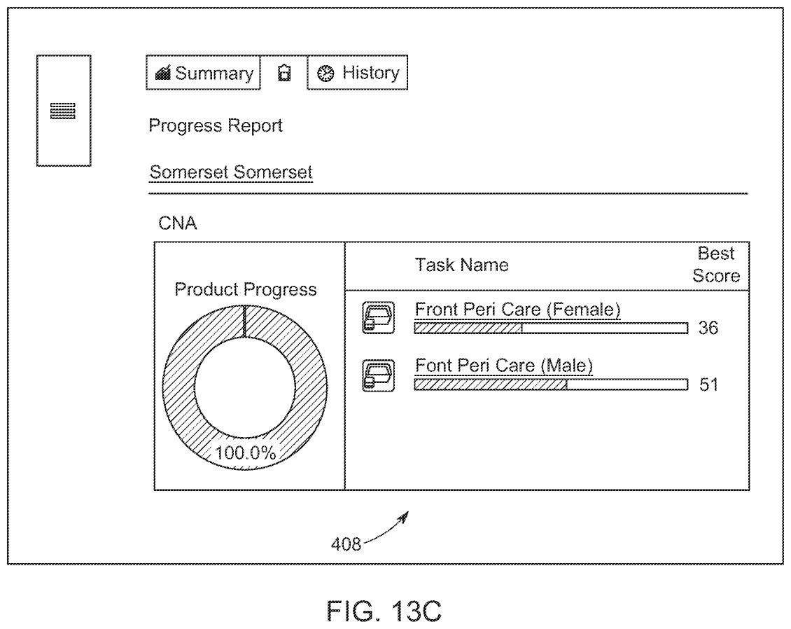

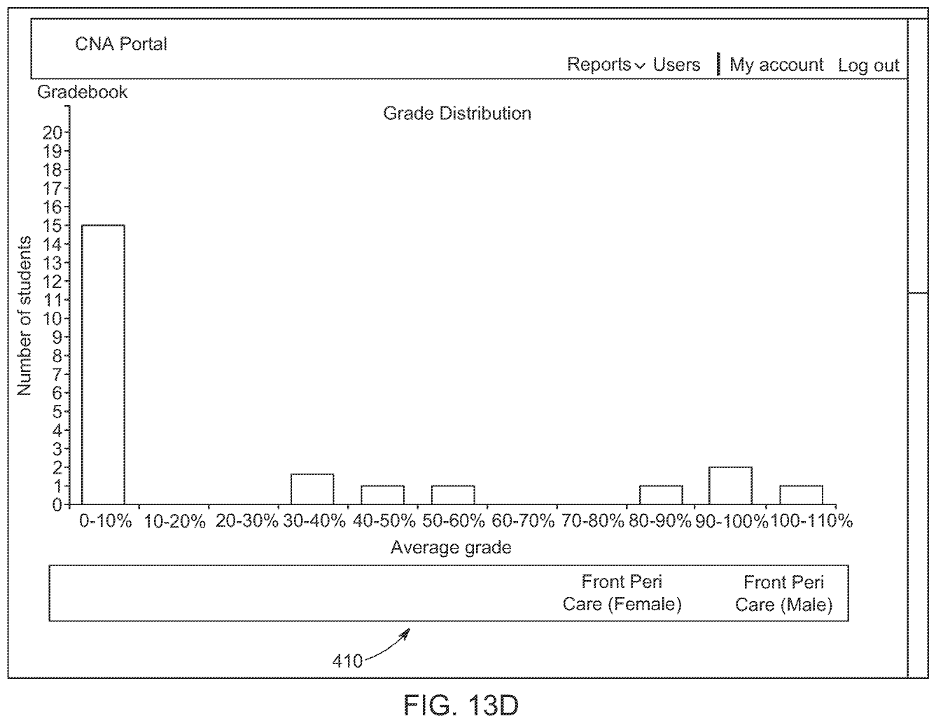

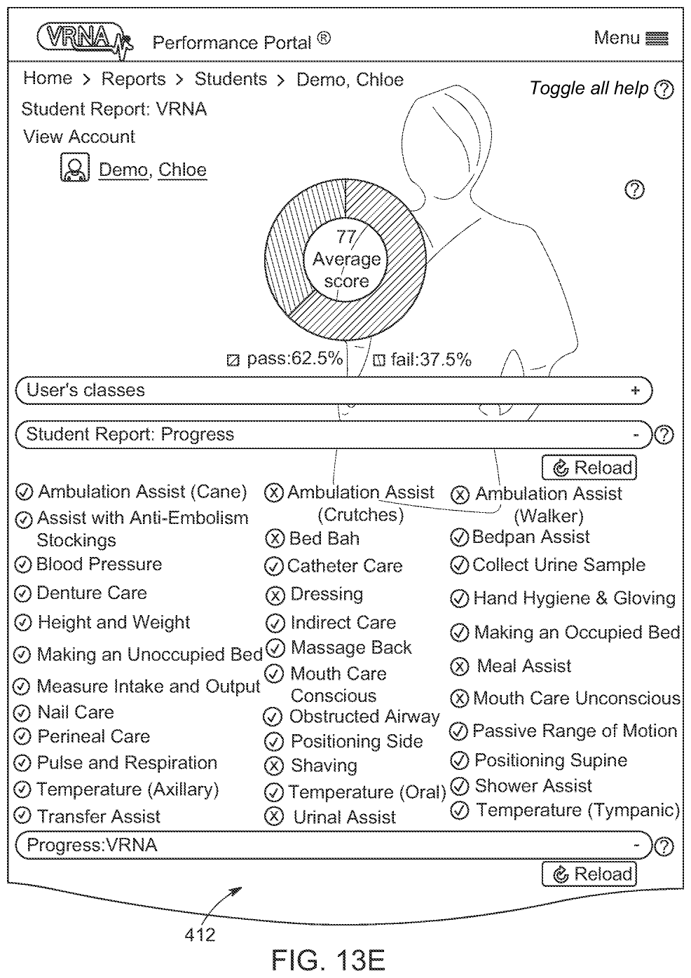

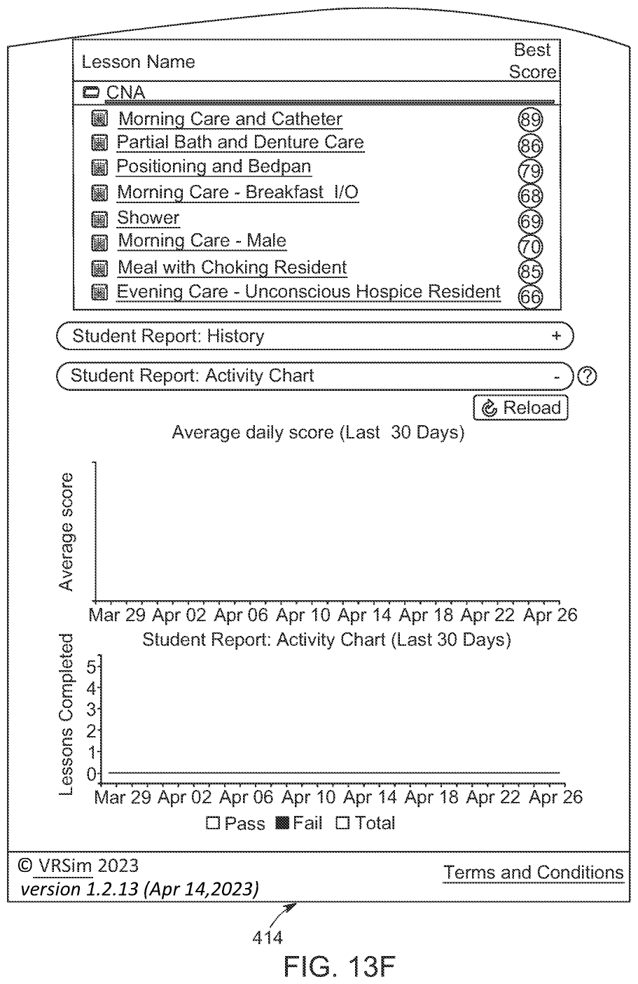

In one embodiment, scoring and/or rewards are stored by the VRNA healthcare simulator 20, for example, within the aforementioned performance data 158, individual and group scores 160 and 162, as compared to performance criteria 156 of the data storage device 150 for one or more trainee/operators 10. In one embodiment, the scoring and/or rewards may be downloaded and transferred out of the training simulator 20 such as, for example, via a USB port (e.g., port 58) on the computing device 52. In one embodiment, scoring and/or rewards for one or more trainees (e.g., the operators 10) may be shared electronically, for example, included in electronic mail messages, posted on a portal accessible by one or more healthcare facilities or the like, websites and bulletin boards, and over social media sites. In one embodiment, shown in GUIs 400, 404, 408, and 412 of FIGS. 13A, 13B, 13C, and 13D, respectively, the training simulator 20 provides a reporting feature wherein a User List (GUI 400 of FIG. 13A) including user statistics, shown generally at 402, group and individual scores 405 in list 405A and bar chart 405B form (GUI 404 of FIG. 13B) and Progress Reports (GUI 408 of FIG. 13C), and a Grade Distribution (GUI 410 of FIG. 13D), may be invoked and viewed. In one embodiment, illustrated on GUI 400 of FIG. 13A, a Reports feature 403 may be invoked to launch reports depicting performance of a healthcare trainee within the VRNA healthcare simulator 20. In one embodiment, one or more of the operators 10 may provide records of scores 405 and/or achieved levels of skill and/or certification as, for example, a portfolio of certifications and/or sample performances that can be viewed and evaluated by potential employers and the like. In one embodiment, shown in GUIs 412 and 414 of FIGS. 13E and 13F, a Performance Portal™ website may be invoked by the processing system 50 of the VRNA healthcare simulator 20 to access the score 405 and various reports of trainees' progress in obtaining and maintaining requisite skills. In one embodiment, the user/healthcare trainee's scores 405 are stored within the learning management system (LMS) 170 and provided as output for the healthcare trainee, teacher, or the like, to track the trainee's progress.

In one embodiment, a healthcare trainee may “earn” an award, commendation, and/or badge when the trainee's score in performing an activity meets or exceeds one or more predetermined thresholds. As such, the awards, commendations, and badges are in recognition for superlative performance, e.g., performance at or above one or more predetermined performance thresholds. In one embodiment, the performance thresholds may be set in accordance with, for example, institutional, state, or federal competency requirements as well as other regulatory and/or certifying agencies or the like. In one embodiment, trainees can upload and publish their scores 405 via the network 90 to, for example, social networking websites such as, for example, Facebook®, Twitter®, or the like. The publication is seen to enhance trainee interest, engagement and, further, foster a level of competition that may drive trainees to build advanced skills in order to obtain a “leader” position among his/her classmates and/or peers.

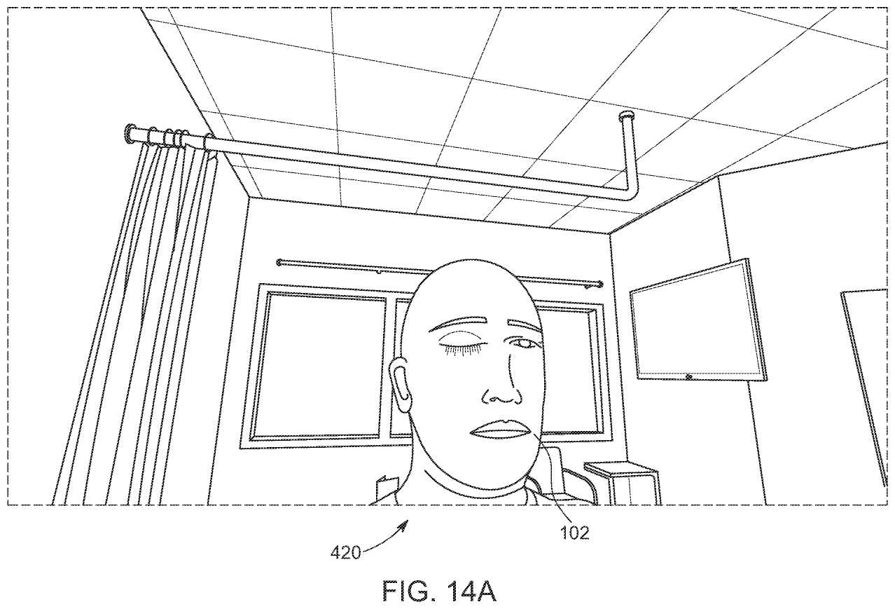

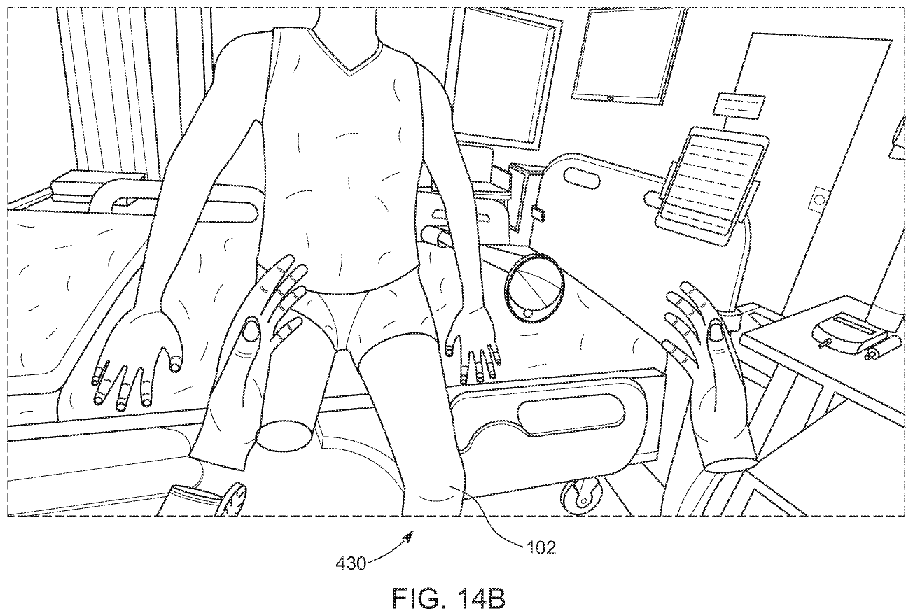

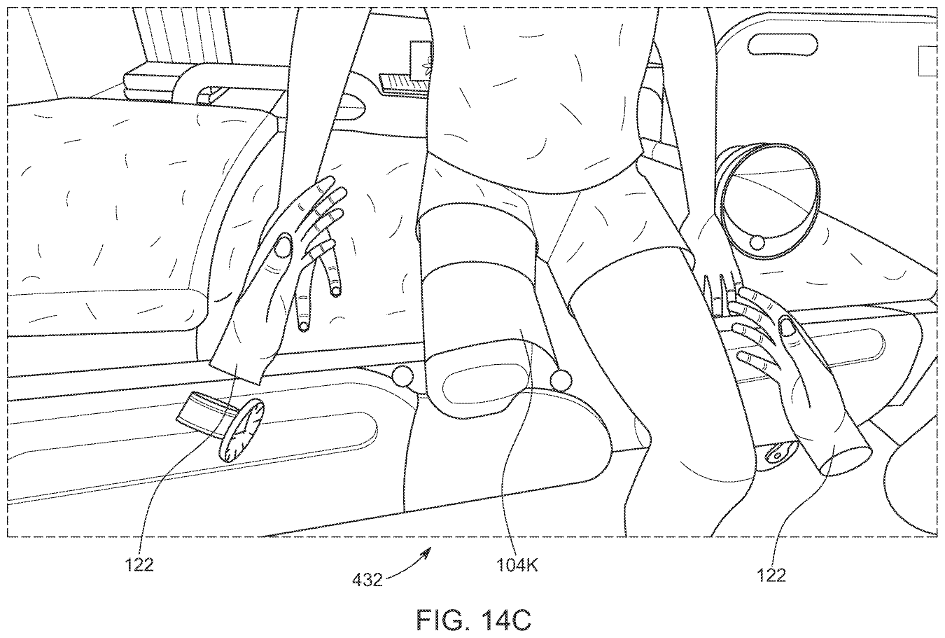

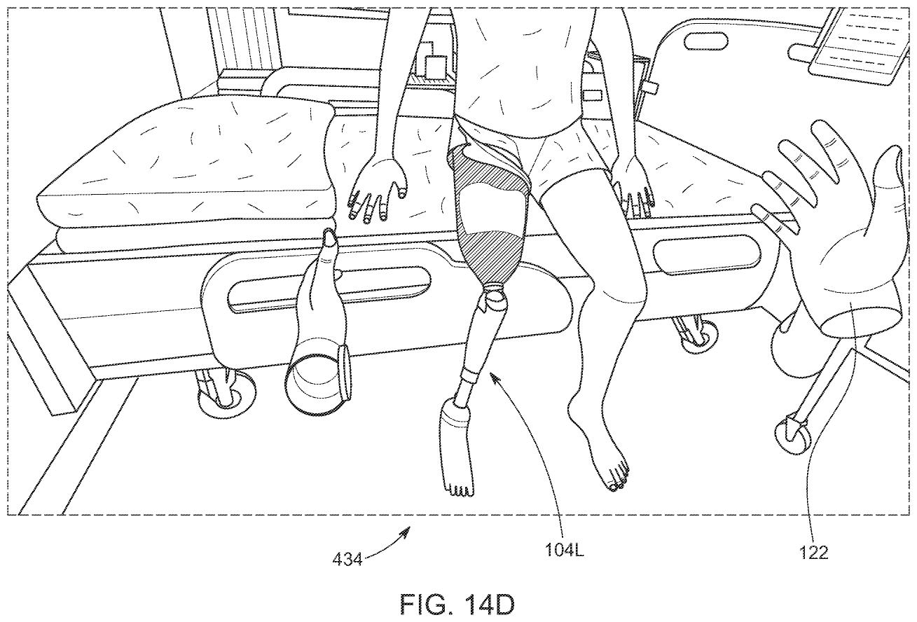

As noted above, it is within the scope of the present invention of the administer of the VRNA simulator 20, the instructor or certification agent 12, and/or the operator or user 10 to selectively vary characteristics or physical features of the simulated resident or patient 102 such as gender, hair, skin tone, skin color, height, weight, and the like, clothing or medical gown worn by the patient 102, medical condition including mental and/or physical conditions, symptoms and/or disabilities of the resident or patient 102 such as, for example, height, weight, patients having an amputated limb or limbs, physical deformities, injuries, wounds, or other medical illnesses, diseases, handicaps, and/or special health care needs, and the like. For example, in one embodiment as illustrated in FIGS. 14A and 14B, GUI 420 depicts a patient 102 as missing one of his eyes and GUI 430 depicts a patient 102 as missing one of his legs, e.g., as an amputee. As shown in FIGS. 14C and 14D, it is within the scope of the present invention to provide healthcare training examples to address these patients with special conditions. For example, in one embodiment illustrated in GUI 432 of FIG. 14C, the VRNA simulator 20 presents the amputee patient 102 and the operator 10 manipulates the avatar 120 and/or its virtual hands 122 with the one or more controllers 60 to retrieve a sock 104K and apply it to cover the patient's residual limb or stump. As illustrated in GUI 434 of FIG. 14D, once the sock 104K is applied, the operator 10 manipulates the avatar 120 and/or its virtual hands 122 with the one or more controllers 60 to retrieve a prosthetic leg 104L and attaches, or assists the patient 102 in attaching, the prosthetic leg 104L to the patient's residual limb.

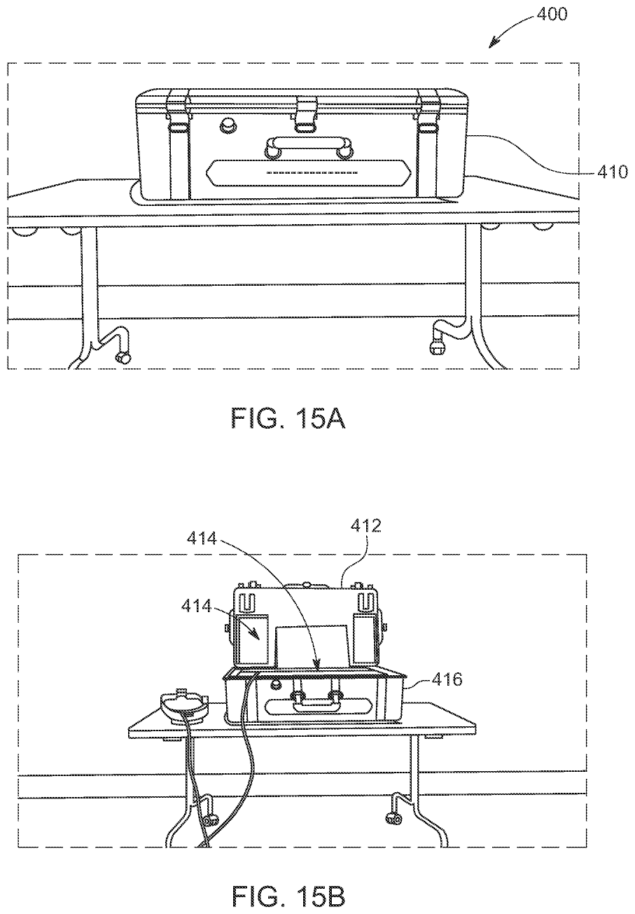

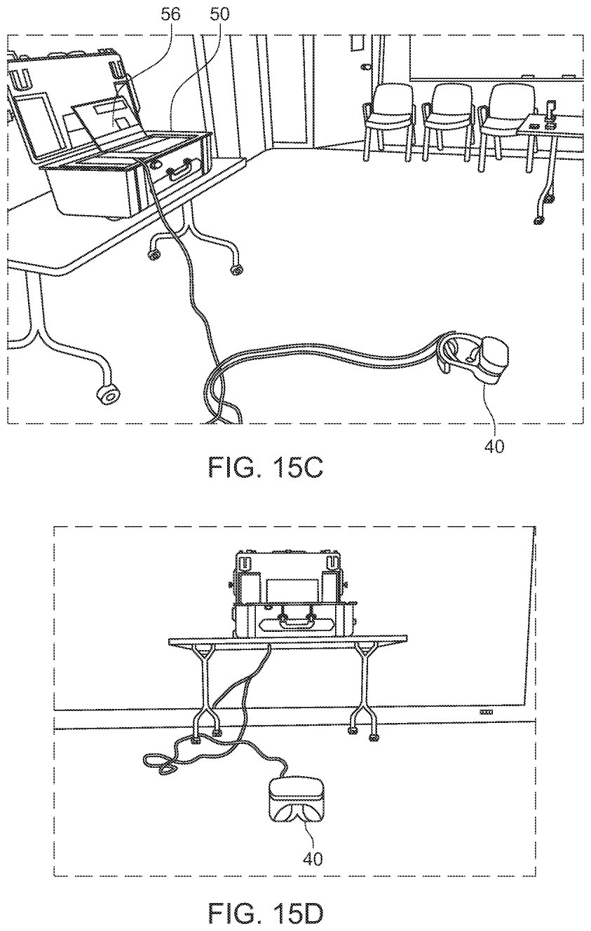

In one aspect of the present invention, the VRNA healthcare training simulator 20 is portable (e.g., transferable) as a self-contained modular assembly 400 (FIG. 15A). The modular assembly 400 includes case or trunk 410 having a cover 412 that is selectively coupled and uncoupled from a housing 416 (FIG. 15B). Once the cover 412 is uncoupled and disposed away from the housing 416, one or more interior chambers or compartments 414 within an interior of the housing 416 are revealed (FIG. 15B). As illustrated in FIG. 15B, components of the healthcare training simulator 20 may be stored within the compartments 414 for storage and/or transportation. For example, the HMDU 40 and one or more controllers 60 are stored in compartments 414. Similarly, external devices such as the computing device 50, speakers 55, and the display 56 are also stored within the compartments 414. In one aspect of the invention as illustrated in FIGS. 15C and 15D, the portability of the healthcare training simulator 20 supports training outside a formal training environment. For example, the operators 10 may initially utilize the simulator 20 at home or at their workplace without supervision by the instructor 12 as a mechanism for early exposure to the skills needed to successful perform healthcare procedures at acceptable levels. Once the operator 10 achieves a basic understanding of the skills, training with the instructor 12 can focus upon the operator's demonstrated weaknesses while only reinforcing demonstrated strengths. This focused and/or targeted training is seen as an advantage provided by the healthcare training simulator 20 as it concentrates instruction upon demonstrated strengths and weaknesses to maximize instructor-student/trainee interaction. As can be appreciated the demonstrated strengths and weaknesses can be shown to the instructor 12 at an individual trainee level as well as a team or class of trainees' level. In addition to use as an initial introduction to skills, the portability provides an ability for an operator having continued deficiencies in one or more skills to take the simulator 20 away from the training environment (e.g., to his/her home or workplace) and focus upon specific areas of concerns outside the scheduled training time.

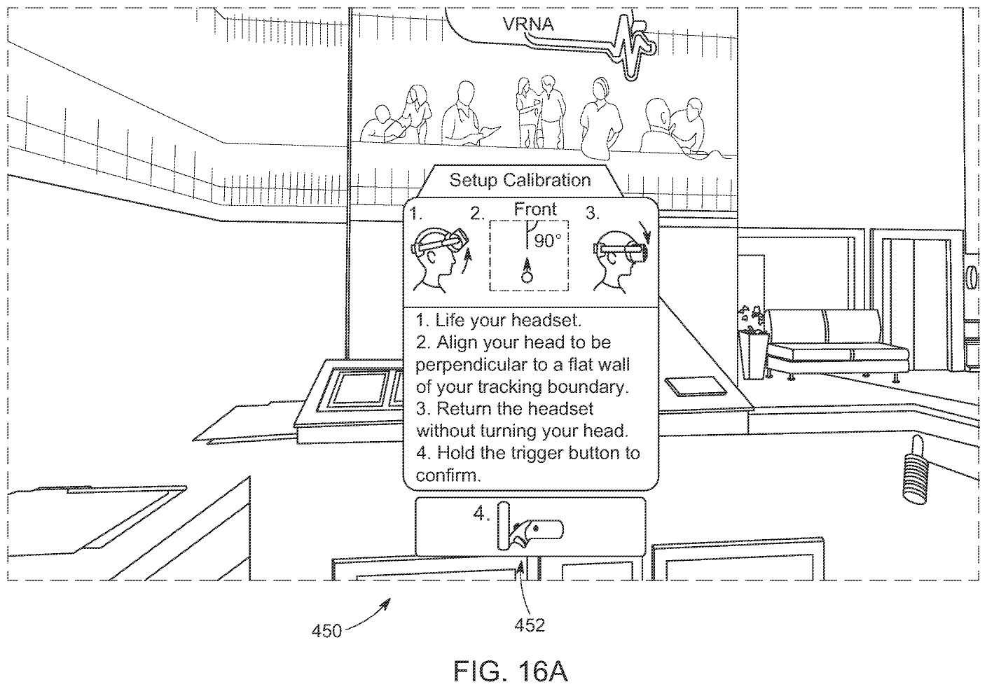

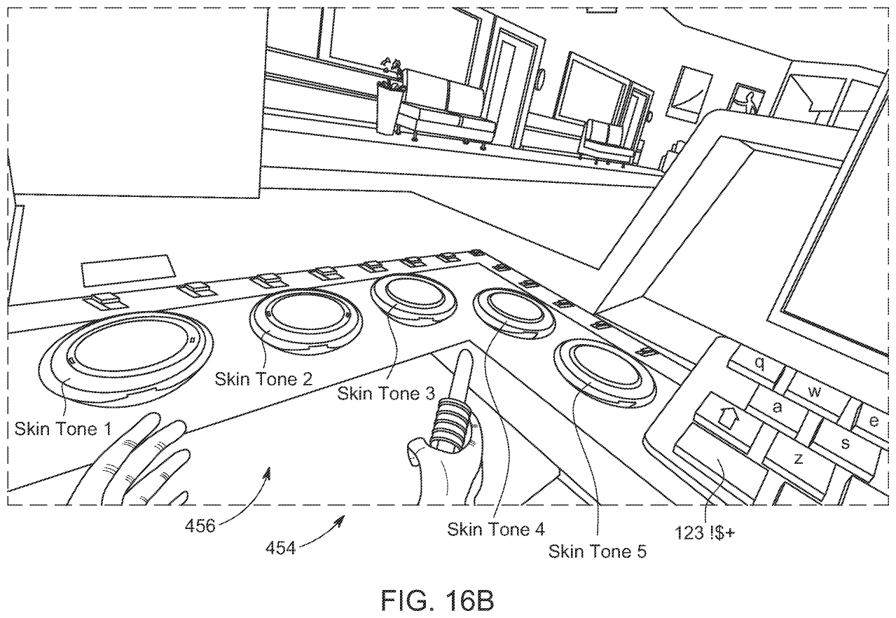

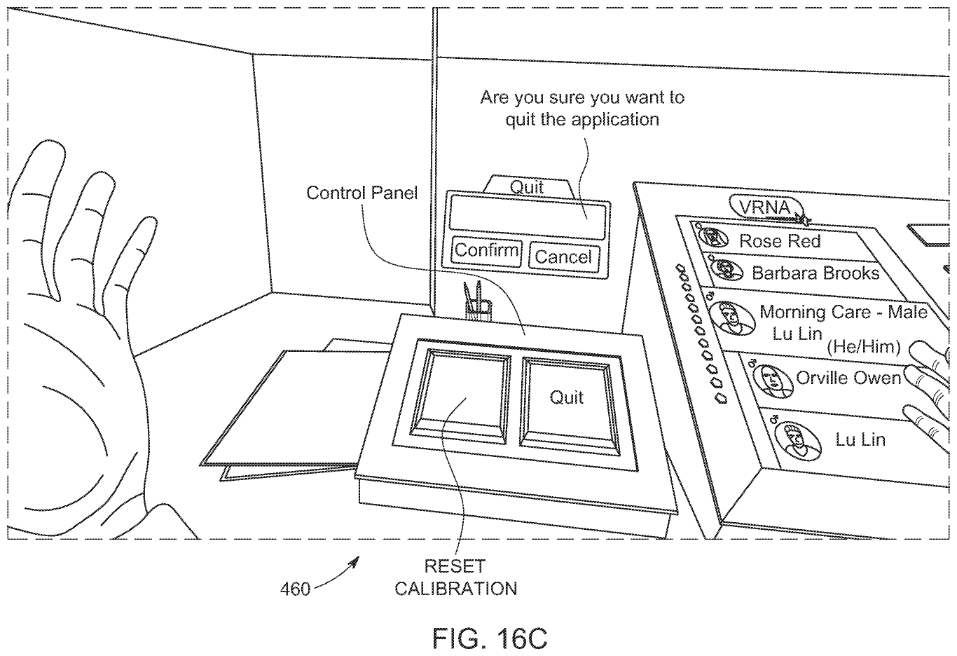

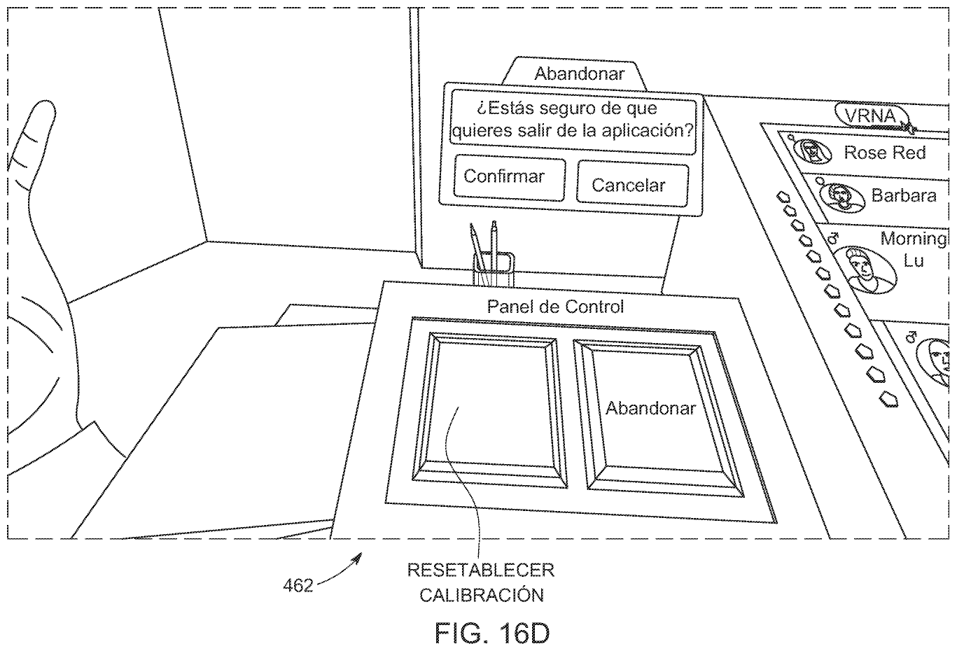

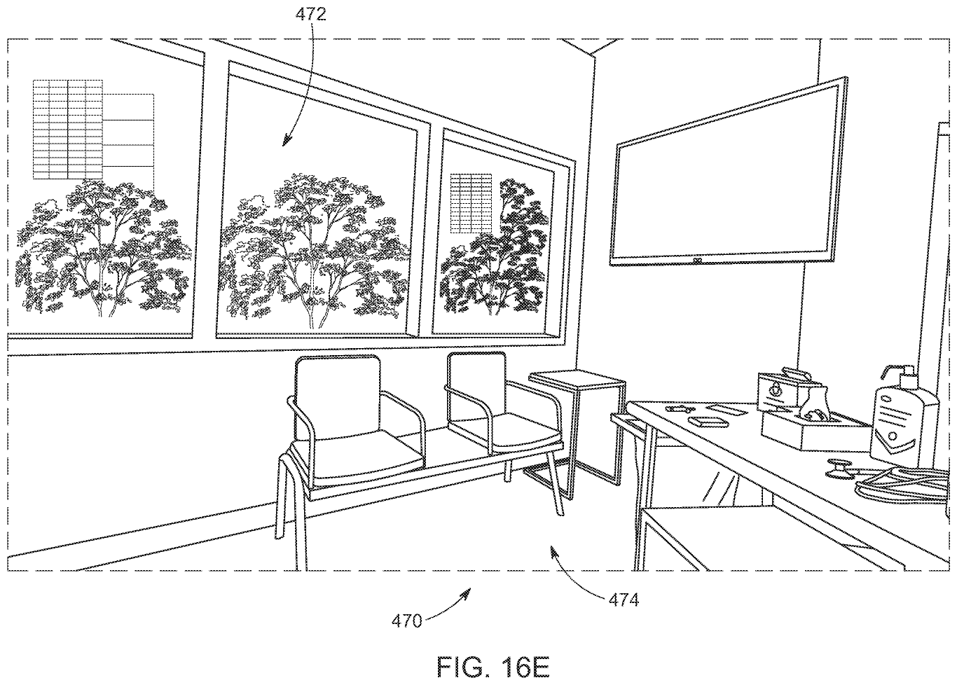

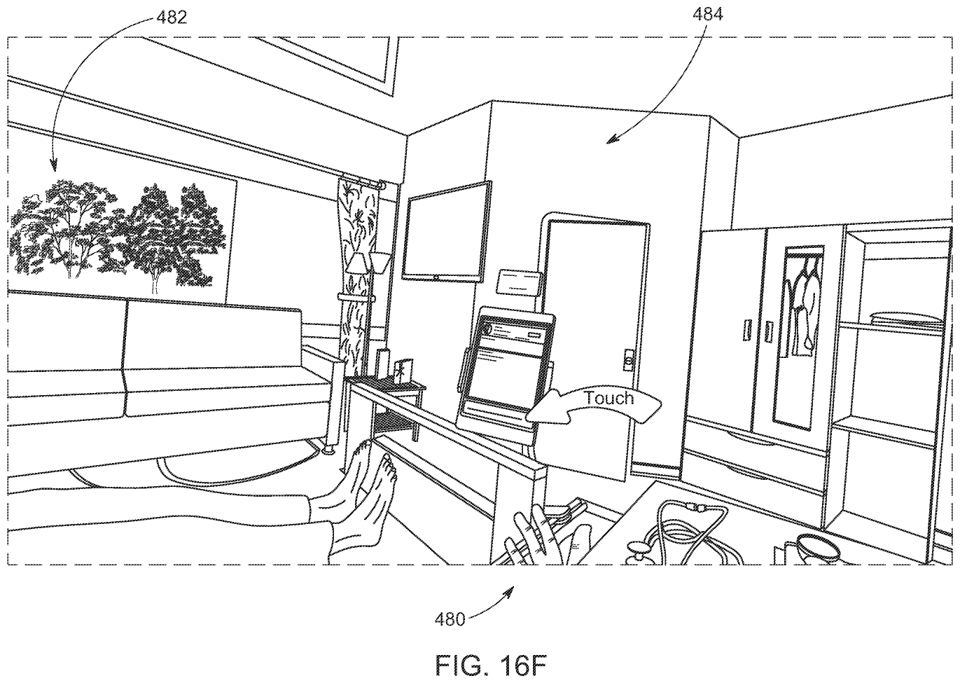

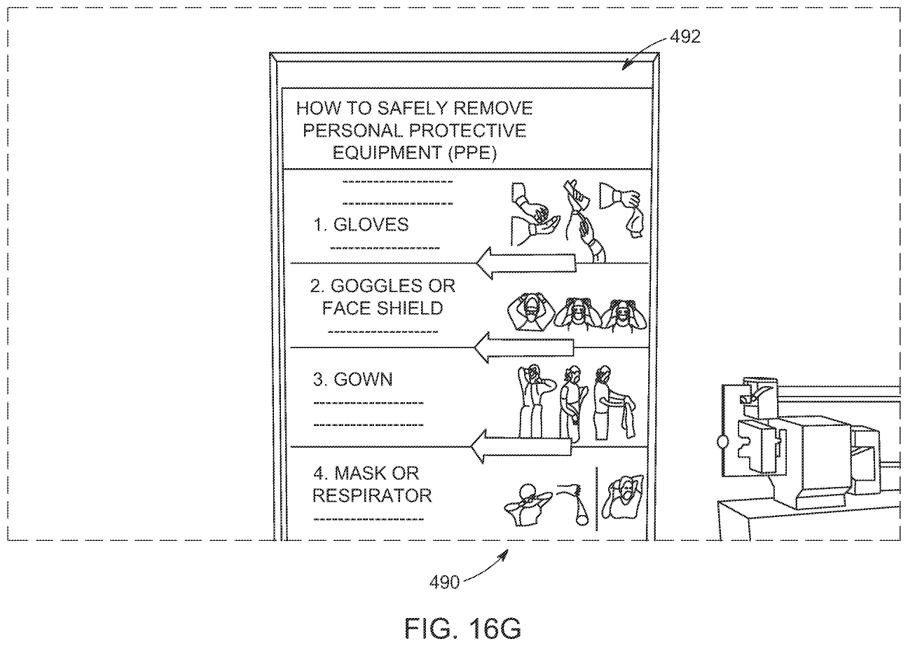

In one aspect of the present invention, the VRNA healthcare training simulator 20 is customizable (e.g., modifiable and/or adjustable) to assign particular characteristics of an operator, e.g., height, spoken language, and the like, and/or environmental settings where healthcare is to be performed, e.g., urban versus rural setting and a particular healthcare facility's room configurations (e.g., single versus multiple resident/patient occupancy, equipment present, display of instruction, informational, and/or hazard/warning postings or displays (e.g., specific PPE required for access)). For example, in one embodiment, the VRNA healthcare training simulator 20 includes a configuration mode, depicted in GUI 450 of FIG. 16A. In one embodiment, the configuration mode includes a setup calibration for the HMDU 40 worn by the user, e.g., operator 10, shown generally at 452. As shown in GUI 454 of FIG. 16B, the VRNA healthcare training simulator 20 includes a setting mode where an operator or administrator may assign or modify characteristics of operator avatars 120 and/or residents/patients 102 such as, for example, vary their skin tone, height, weight, medical or health conditions, by, for example, selecting from system defined alternatives, shown generally at 456 for skin tone variations. As shown in GUIs 460 and 462 of FIGS. 16C and 16D, respectively, the VRNA healthcare training simulator 20 includes a setting to define a language for display and entry of data and information, and messaging to and by operators. For example, GUIS s 460 and 462 include, for example, data and information displayed in English and Spanish languages. As should be appreciated, it is within the scope of the present invention to permit display and entry of data and information in a plurality of different languages as training needed and/or desired to facilitate use of the VRNA healthcare training simulator 20 and training of healthcare practitioners. As shown in GUIs 470 and 480 of FIGS. 16E and 16F, respectively, the VRNA healthcare training simulator 20 includes a setting to define the environmental settings where healthcare is to be performed. As shown in GUI 470 of FIG. 16E, the environment is exhibited as an urban, e.g., city, office setting, as shown generally at 472 and 474, respectively as compared to GUI 480 of FIG. 16F, where the environment is exhibited as a rural, residential setting, as shown generally at 482 and 484, respectively. As shown in GUI 490 of FIG. 16G, one or more of the healthcare training environments depicted in the VRNA healthcare training simulator 20 may include environmental healthcare instructions and/or messaging, shown at 492, in one or more languages. As should be appreciated, it is within the scope of the present invention to implement a plurality of different environmental settings to simulate where healthcare is provided and to facilitate training within a familiar facility in which healthcare will actually be rendered by the healthcare practitioners being trained with the VRNA healthcare training simulator 20 to, for example, provide a more realistic training experience.

While the invention has been described with reference to various exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A simulator for skill-oriented training of a healthcare task, the simulator comprising:
a head-mounted display unit (HMDU) wearable by an operator operating the simulator, the HMDU having at least one camera, at least one speaker, at least one display device, and at least one HMDU sensor, the at least one camera, the at least one speaker, and the at least one display device providing visual and audio output to the operator;
one or more controllers operable by the operator, the one or more controllers each having at least one controller sensor, the at least one controller sensor and the at least one HMDU sensor each cooperating to measure and to output one or more signals representing spatial positioning, angular orientation, speed and direction of movement data of the one or more controllers relative to a patient as the operator performs a healthcare task;
a data processing system operatively coupled to the HMDU and the one or more controllers, the data processing system including a processor and memory operatively coupled to the processor with a plurality of executable algorithms stored therein, the processor is configured by the executable algorithms to:
determine coordinates of a position, an orientation, and a speed and a direction of movement of the one or more controllers in relation to the patient as the operator takes actions to perform the healthcare task based on the one or more signals output from the at least one HMDU sensor and the at least one controller sensor of each of the one or more controllers;
model the actions taken by the operator to perform the healthcare tasks to determine use of healthcare equipment and supplies and changes in condition of the patient, reaction of the patient, and the used healthcare equipment and supplies in relation to the actions taken;
render the patient, the used healthcare equipment and supplies, the condition of the patient, the reaction of the patient, changes to the condition of the patient, changes to the used healthcare equipment and supplies, and sensory guidance as to the performance of the healthcare tasks from the actions taken by the operator in a three-dimensional virtual training environment; and
simulate in real-time the three-dimensional virtual training environment depicting the rendered patient, the rendered reaction of the patient, the rendered used healthcare equipment and supplies, the rendered changes to the condition of the patient, the rendered changes to the used healthcare equipment and supplies, and the rendered sensory guidance as the operator performs the healthcare task in the training environment;
wherein the rendered patient, the rendered reaction of the patient, the rendered used healthcare equipment and supplies, the rendered changes to the condition of the patient, the rendered changes to the used healthcare equipment and supplies, and the rendered sensory guidance are exhibited in near real-time to the operator within the training environment on the at least one display device of the HMDU to provide in-process correction and reinforcement of preferred performance characteristics as the operator performs the healthcare task; and
wherein the rendered sensory guidance includes a plurality of visual, audio and tactile indications of performance by the operator as compared to optimal values for performance.

2. The simulator of claim 1, further includes an avatar or portion thereof, manipulated and directed by the operator with the one or more controllers to take the actions to perform the healthcare task in the three-dimensional virtual training environment.

3. The simulator of claim 2, wherein the portion of the avatar includes virtual hands.

4. The simulator of claim 1, wherein the operator further includes a plurality of operators undertaking the skill-oriented training as a group cooperating to perform the healthcare task within the three-dimensional virtual training environment.

5. The simulator of claim 1, wherein the operator is one of a medical professional and an individual providing home health aid.

6. The simulator of claim 5, wherein the medical professional includes at least one of an emergency medical technician (EMT), a licensed practical nurse (LPN), and a certified nursing assistant, nurse's aid, or a patient care assistant referred to herein as a CNA.

7. The simulator of claim 1, wherein a path of travel of the operator performing the healthcare tasks is modeled, based on at least one of a position, orientation, speed and direction of movement of the HMDU and the one or more controllers.

8. The simulator of claim 1, wherein the visual indications of performance include an indication, instruction, and/or guidance of the optimal values for preferred performance of the healthcare task currently being performed by the operator.

9. The simulator of claim 1, wherein the audio indications of performance include an audio tone output by the at least one speaker of the HMDU.

10. The simulator of claim 9, wherein the audio tone is a reaction by the patient to the healthcare task currently being performed by the operator.

11. The simulator of claim 1, further including a display device operatively coupled to the data processing system such that an instructor may monitor the performance by the operator of the healthcare task.

12. The simulator of claim 1, wherein the visual indications include a score or grade for the operator in the performance by the operator of the healthcare task as compared to a set of performance criteria defining standards of acceptability.

13. The simulator of claim 12, wherein the score or grade is a numeric value based on how close to optimum the operator's performance is to the set of performance criteria.

14. The simulator of claim 12, wherein the score or grade further includes rewards including certification levels and achievements highlighting the operator's results as compared to the set of performance criteria and to other operators.

15. The simulator of claim 14, wherein the score or grade and rewards for one or more of the operators are at least one of shared electronically, posted on a website or bulletin board, and over social media sites.

16. The simulator of claim 1, wherein the data processing system is further configured to provide a review mode for evaluating the operator's performance of the healthcare task.

17. The simulator of claim 16, wherein when in the review mode the data processing system is further configured to provide reports of the operator's performance.

18. The simulator of claim 16, wherein when in the review mode the data processing system is further configured to provide the review mode to at least one of the operator of the controller, an instructor overseeing the skill-oriented training, and other operators undergoing the skill-oriented training.

19. The simulator of claim 1, wherein the simulator is portable as a self-contained modular assembly.

20. The simulator of claim 1, wherein the data processing system is further configured to provide one or more modes for assigning characteristics of at least one of the operator, the patient, and the environmental setting where the healthcare task is performed.
